(19) United States
(12) Patent Application Publication
(10) Pub. No.: US 2016/0030323 A1
(43) Pub. Date: Feb. 4, 2016

(54) METHODS AND MATERIALS FOR PROVIDING TEETH WITH A WHITE APPEARANCE

(71) Applicant: SAFEWHITE, INC., Dublin, OH (US)

(72) Inventors: Richard Simon Brody, Columbus, OH (US); Thomas Joel Zupancic, Powell, OH (US); Nicole Hoefer, Bexley, OH (US); Uday B. Sandbhor, Hilliard, OH (US)

(73) Assignee: SAFEWHITE, INC., Dublin, OH (US)

(21) Appl. No.: 14/776,524

(22) PCT Filed: Mar. 14, 2014
(86) PCT No.: PCT/US14/28233

§ 371 Date: Sep. 14, 2015

Related U.S. Application Data
(60) Provisional application No. 61/785,966, filed on Mar. 14, 2013.
(60) Provisional application No. 61/903,671, filed on Nov. 13, 2013.

(51) Int. Cl. A61K 8/64; A61Q 11/00; A61K 8/24; A61K 8/29

(52) U.S. Cl. CPC A61K 8/64; A61K 8/29; A61Q 11/00; A61K 8/24; A61K 2800/28; A61K 2800/42; A61K 2800/592; A61K 2800/413

(57) ABSTRACT

This document provides methods and materials for delivering compounds to teeth and methods and materials for providing teeth with a whiter appearance. For example, methods and materials are provided for contacting teeth with one or more adhesive molecules (e.g., mussel adhesive polypeptides or polymer containing a plurality of 3,4-dihydroxyphenyl-L-alanine residues) in combination with one or more fluorescence emitting polypeptides (e.g., a blue fluorescent protein (BFP)) and/or whitening particles to provide the teeth with a whiter appearance.

### CROSS-REFERENCE TO RELATED APPLICATIONS

This application claims priority to U.S. Application Ser. No. 61/903,671, filed Nov. 13, 2013, and to U.S. Application Ser. No. 61/785,966, filed on Mar. 14, 2013, the disclosures of which are incorporated by reference in their entirety.

### BACKGROUND

1. Technical Field

This document relates to methods and materials for delivering compounds to teeth using one or more adhesive molecules. This document also relates to providing teeth with a white appearance. For example, this document relates to methods and materials for contacting teeth with one or more adhesive molecules (e.g., one or more mussel adhesive polypeptides) in combination with one or more polypeptides (e.g., fluorescence emitting polypeptides such as blue fluorescent protein or non-fluorescent polypeptides such as collagen or albumin) and/or one or more whitening particles or other compounds to provide the teeth with a whiter appearance.

2. Background Information

In general, white teeth are considered cosmetically desirable. Teeth, however, can become discolored in the absence of intervention. The tooth structure that is generally responsible for presenting a stained appearance is the enamel layer. Several factors can contribute to enamel discoloration. For example, the formation of plaque and tartar matrices on the tooth surface can entrap stains, thereby leading to enamel discoloration.

Over-the-counter tooth whitening preparations have been developed to address the cosmetic preference of many to restore luster to tooth enamel discolored by surface entrapped materials. While all dentifrices and mouthwashes contain some cleaning and polishing agents, some enamel deposits or other alterations to enamel appearance become intractable to being fully removed, oxidized, or re-colored by these agents under normal use conditions. Smokers often develop discolored enamel because the tars and particulates in exhaled cigarette smoke collect on the teeth. In some case, foods and drinks (e.g., tea) can stain or discolor tooth enamel.

### SUMMARY

This document provides methods and materials for delivering compounds to the teeth or mouth (e.g., the teeth or mouth of a human) using adhesive molecules alone or in combination with one or more polypeptides (e.g., fluorescence-emitting polypeptides or non-fluorescent polypeptides). For example, the methods and materials described herein can be used to deliver nucleic acids, polypeptides, therapeutic agents, whitening particles, or remineralization particles to teeth. In some cases, delivery of the compounds improves the white appearance of the teeth and/or binds remineralization particles to the teeth.

This document also provides methods and materials for providing teeth with a whiter appearance. For example, this document provides methods and materials for contacting teeth with one or more adhesive molecules (e.g., one or more mussel adhesive polypeptides) in combination with one or more fluorescence emitting polypeptides (e.g., a blue fluorescent protein (BFP)) and/or one or more whitening particles to provide the teeth with a whiter appearance. As described herein, adhesive molecules can be applied to teeth in combination with fluorescence emitting polypeptides such as BFP polypeptides or non-fluorescent polypeptides and/or whitening particles such as hydroxyapatite or titanium dioxide particles. The adhesive molecule can include a plurality of 3,4-dihydroxyphenyl-L-alanine (DOPA) residues and can have the ability to interact with or bind to a tooth, a tooth component such as enamel, hydroxyapatite, or acquired dental pellicle, or inorganic dental materials such as crowns, caps, braces, or fillings. The methods and materials described herein can be used on dry teeth or on wet teeth. For example, the methods and materials described herein can be useful under typical conditions found in the mouth, such as the presence of saliva on the teeth and food, and under the duress of brushing or eating.

The fluorescence emitting polypeptides can emit fluorescence at a particular wavelength. In the case of BFP polypeptides, the BFP polypeptides can emit fluorescence in the range of about 440 nm to about 500 nm (e.g., between about 450 nm and about 490 nm), which when emitted from teeth give the teeth a white appearance. This white appearance can occur even when the underlying teeth are not naturally that white. For example, the methods and materials provided herein can allow a person to have white appearing teeth even though the teeth may be stained or have been discolored due to a chemical such as tetracycline. Thus, white appearing teeth can be obtained using the methods and materials provided herein without harsh bleaching (e.g., without dental bleaching treatments such as those involving hydrogen peroxide or carbimide peroxide) or de-staining techniques. The methods and materials described herein provide teeth with a natural appearance as the fluorescent polypeptides mimic the natural fluorescence of teeth and help to preserve translucence by not being entirely opaque.

In one aspect, this document features a method of delivering a compound to teeth (e.g., human teeth). The method include applying to teeth a composition comprising, or consisting essentially of, an adhesive molecule, a non-fluorescent polypeptide, and the compound (e.g., polypeptide, a nucleic acid, a fluorescent moiety, a therapeutic agent, a whitening particle, or a remineralization particle), the adhesive molecule comprising a plurality of DOPA residues and having the ability to interact with or bind to a tooth, a tooth component, or inorganic dental material. The adhesive molecule can be conjugated to a non-fluorescent polypeptide such as collagen or albumin. The conjugate of the adhesive molecule and the non-fluorescent polypeptide can be non-aggregating.

In general, one aspect of this document features a method for altering the appearance of teeth. The method comprises, or consists essentially of, applying to teeth (e.g., to dry human or animal teeth, to wet human or animal teeth, or to human or animal teeth under typical conditions found in the mouth) an adhesive molecule that contains a plurality of DOPA residues and a fluorescence emitting polypeptide (e.g., a BFP polypeptide), wherein the adhesive molecule has the ability to interact with or bind to a tooth, a tooth component, or inorganic dental materials, and wherein fluorescence emitted from the fluorescence emitting polypeptide alters the appearance of the teeth. The adhesive molecule and fluorescence emitting polypeptide can be applied sequentially or applied together. For example, the adhesive molecule and fluorescence emitting polypeptide can be applied in the form of a conjugate, in which the adhesive molecule and fluorescence emitting polypeptide are linked together with chemical crosslinking reagents or can be applied as a chimeric polypeptide containing both adhesive and fluorescent properties. The method further can include altering the appearance of the teeth such that the teeth appear whiter. The method further can include applying whitening particles (e.g., nanoparticles or microparticles) to the teeth. For example, the whitening particles can be bound to a conjugate containing an adhesive molecule and a fluorescent emitting polypeptide, and the complex containing the whitening particles and conjugate can be applied to the teeth. The whitening particles can include hydroxyapatite, silicon dioxide, titanium dioxide, or zinc oxide. Such particles can enhance the whiter appearance of the fluorescing teeth under certain conditions and allow the appearance to be more consistent over the surface of the teeth.

The adhesive molecule, fluorescence emitting polypeptide or non-fluorescent polypeptide, and whitening or remineralization particles can be present within tooth paste, mouth rinse, mouth wash, application gel, chewing gum, or ingested substance and the applying step can include applying (e.g., directly applying) the tooth paste, mouth rinse, mouth wash, application gel, chewing gum, or ingested substance to teeth. In some cases, the mouth wash, mouth rinse, application gel, chewing gum, or ingested substance are in contact with teeth or tooth component for a period of time sufficient for the teeth or tooth component to be saturated.

In another aspect, this document features a method for altering the appearance of teeth. The method comprises, or consists essentially of, applying (e.g., to dry human or animal teeth, to wet human or animal teeth, or to human or animal teeth under typical conditions found in the mouth) an adhesive molecule and a whitening particle (e.g., nanoparticles) or remineralization particle to teeth, the adhesive molecule comprising a plurality of DOPA residues and having the ability to interact with or bind to a tooth, a tooth component, or inorganic dental materials. The appearance of the teeth can be altered (e.g., make the teeth appear whiter) and/or remineralization particles can be bound to the teeth. The adhesive molecule and whitening or remineralization particles can be applied sequentially or applied together. The whitening particle can include hydroxyapatite, titanium dioxide, silicon dioxide, or zinc oxide particles. The remineralization particle can include hydroxyapatite or amorphous calcium phosphate particles. The adhesive molecule can include a polymethacrylate polymer comprising the plurality of DOPA residues. The adhesive molecule can include one or more mussel adhesive polypeptides. The adhesive molecule can include a polymer comprising or consisting essentially of a plurality of lysine residues and the plurality of DOPA residues. The adhesive molecule can include polymer comprising or consisting essentially of a plurality of lysine residues, a plurality of glycine residues, and the plurality of DOPA residues. The polymer can have the amino acid sequence set forth in SEQ ID NO:21 or SEQ ID NO:22.

This document also features a composition comprising, or consisting essentially of, a fluorescence emitting polypeptide and an adhesive molecule comprising a plurality of DOPA residues, the adhesive molecule having the ability to interact with or bind to a tooth, a tooth component, or inorganic dental material. The composition further can include whitening particles such as titanium dioxide, silicon dioxide, hydroxyapatite, or zinc oxide particles. The adhesive molecule can be a polymer comprising or consisting essentially of a plurality of lysine residues and the plurality of DOPA residues. The adhesive molecule can include polymer comprising or consisting essentially of a plurality of lysine residues, a plurality of glycine residues, and the plurality of DOPA residues. The adhesive molecule can be one or more mussel adhesive polypeptides. The fluorescence emitting polypeptide can be conjugated to the mussel adhesive polypeptide. The adhesive molecule can be a polymethacrylate polymer comprising the plurality of DOPA residues. The fluorescence emitting polypeptide can be conjugated to the polymethacrylate polymer.

In another aspect, this document features a composition comprising, or consisting essentially of, an adhesive molecule and a whitening particle or remineralization particle, the adhesive molecule comprising a plurality of DOPA residues and having the ability to interact with or bind to a tooth, a tooth component, or inorganic dental material. The adhesive molecule can be a polymer comprising or consisting essentially of a plurality of lysine residues and the plurality of DOPA residues. The adhesive molecule can include polymer comprising or consisting essentially of a plurality of lysine residues, a plurality of glycine residues, and the plurality of DOPA residues. The adhesive molecule can be one or more mussel adhesive polypeptides. The adhesive molecule can be a polymethacrylate polymer comprising the plurality of DOPA residues. The whitening particle can be a silicon dioxide, hydroxyapatite, or zinc oxide particle. The remineralization particle can be a hydroxyapatite or amorphous calcium phosphate particle. The composition further can include a fluorescence emitting polypeptide or a non-fluorescent polypeptide. The fluorescence emitting polypeptide or non-fluorescent polypeptide can be conjugated to the adhesive molecule (e.g., mussel adhesive polypeptide, polymethacrylate polymer, polymer comprising a plurality of lysine and DOPA residues, or polymer comprising a plurality of lysine, glycine, and DOPA residues). The conjugate of the fluorescence emitting polypeptide or the non-fluorescent polypeptide and the adhesive molecule can be non-aggregating. The conjugate of the fluorescence emitting polypeptide or the non-fluorescent polypeptide and the adhesive molecule can be stable under conditions found in the mouth of a human. The fluorescence emitting polypeptide or the non-fluorescent polypeptide and the adhesive molecule can be conjugated at a pH less than 7.0.

In another aspect, this document features a composition comprising, or consisting essentially of, hydroxyapatite particles and an adhesive molecule conjugated to a polypeptide (e.g., a fluorescence emitting polypeptide or a non-fluorescent polypeptide such as collagen or albumin). The adhesive molecule can include a plurality of DOPA residues and have the ability to interact with or bind to a tooth, a tooth component, or inorganic dental material.

This document also features a composition comprising, or consisting essentially of, an adhesive molecule, a non-fluorescent polypeptide, and a compound (e.g. a polypeptide, a nucleic acid, a fluorescent moiety, a therapeutic agent, a whitening particle, or a remineralization particle), the adhesive molecule comprising a plurality of DOPA residues and having the ability to interact with or bind to a tooth, a tooth component, or inorganic dental material. The adhesive molecule can be conjugated to the non-fluorescent polypeptide. The conjugate of the adhesive molecule and the non-fluorescent polypeptide can be non-aggregating.

In any of the compositions described herein, the fluorescence emitting polypeptide can be a BFP polypeptide.

Any of the compositions described herein can be a tooth paste, a mouth rinse, application gel, chewing gum, or ingested substance.

In another aspect, this document features a chimeric polypeptide comprising, or consisting essentially of, an amino acid sequence of 20 or more residues in length of a fluorescence emitting polypeptide and an amino acid sequence of 20 or more residues in length of a mussel adhesive polypeptide. The chimeric polypeptide can comprise a full length fluorescence emitting polypeptide or fragment thereof that is at least about 90 percent identical to the full length fluorescence emitting polypeptide. The chimeric polypeptide can comprise a full length mussel adhesive polypeptide or fragment thereof that is at least about 80 percent identical to the full length mussel adhesive polypeptide. The chimeric polypeptide can comprise the ability to emit fluorescence and the ability to interact with or bind to a tooth, a tooth component, or inorganic dental material.

In any of the methods or compositions described herein, the adhesive molecule can be one or more mussel adhesive polypeptides (e.g., two or more mussel adhesive polypeptides). The mussel adhesive polypeptide can include a mussel foot protein-1 (MFP-1), MFP-3, MFP-5, or a combination thereof For example, the mussel adhesive polypeptide can include a combination of MFP-1 and MFP-5 polypeptides. The mussel adhesive polypeptide can be a chimeric polypeptide comprising (i) an amino acid sequence of MFP-1 and an amino acid sequence of MFP-5 or (ii) an amino acid sequence of MFP-1 and an amino acid sequence of MFP-3. The fluorescence emitting polypeptide can be conjugated to the mussel adhesive polypeptide.

In any of the methods or compositions described herein, the fluorescence emitting polypeptide and the mussel adhesive polypeptide can be part of a chimeric polypeptide that contains an amino acid sequence of the fluorescence emitting polypeptide and an amino acid sequence of the mussel adhesive polypeptide.

In any of the methods or compositions described herein, the adhesive molecule can be a polymer that comprises or consists essentially of a plurality of lysine residues and the plurality of DOPA residues. The polymer can comprise or consist essentially of a plurality of lysine residues, a plurality of glycine residues and a plurality of DOPA residues. The adhesive molecule can have the amino acid sequence set forth in SEQ ID NO:21 or SEQ ID NO:22. The fluorescence emitting polypeptide can be conjugated to the polymer.

In any of the methods or compositions described herein, the adhesive molecule can be a polymethacrylate polymer comprising the plurality of DOPA residues. The fluorescence emitting polypeptide can be conjugated to the polymethacrylate polymer.

In any of the methods or compositions described herein, the fluorescence emitting polypeptide can be one unit of a polymer comprising two or more fluorescence emitting polypeptides. The polymer can be attached to a mussel adhesive polypeptide to form a conjugate, wherein the conjugate is applied to the teeth. For example, the polymer can be conjugated to a mussel adhesive polypeptide and applied to teeth alone or in combination with whitening particles.

In any of the methods or compositions described herein, the adhesive molecule can be one unit of a polymer comprising two or more adhesive molecules. The polymer can be conjugated to a fluorescence emitting polypeptide and applied to teeth alone or in combination with whitening particles.

In any of the methods or compositions described herein, a polymer, where one unit of a polymer comprises an adhesive molecule conjugated to a fluorescence emitting polypeptide, can be applied to teeth alone or in combination with whitening particles.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a listing of a nucleic acid sequence (SEQ ID NO:1) that encodes an exemplary BFP polypeptide (GenBank Accession No. U70497.1; GI No. 1619752).

FIG. 2 is a listing of an amino acid sequence (SEQ ID NO:2) of an exemplary BFP polypeptide (GenBank Accession No. AAB16959.1; GI No. 1619753).

FIG. 3 is a listing of an amino acid sequence (SEQ ID NO:3) of an exemplary BFP polypeptide.

FIG. 4 is a listing of the nucleic acid sequence (SEQ ID NO:4) that encodes an exemplary mussel adhesive polypeptide (GenBank Accession No. AY521220.1; GI No. 41350294).

FIG. 5 is a listing of an amino acid sequence (SEQ ID NO: 5) of an exemplary mussel adhesive polypeptide (GenBank Accession No. AAS00463; GI No. 41350295).

FIG. 6 is a listing of an amino acid sequence (SEQ ID NO: 6) of an exemplary mussel adhesive polypeptide (GenBank Accession No. AAL35297.1; GI No. 17066511).

FIG. 7 is a listing of an acid acid sequence (SEQ ID NO: 7) of an exemplary mussel adhesive polypeptide (GenBank Accession No. ABE01084.1; GI No. 90823165).

FIG. 8 is a listing of an amino acid sequence (SEQ ID NO: 8) of an exemplary mussel adhesive polypeptide (GenBank Accession No. AAF89290.1; GI No. 9587380).

FIG. 9 is a listing of an amino acid sequence (SEQ ID NO: 9) of an exemplary mussel adhesive polypeptide (GenBank Accession No. AAY29129.1; GI No. 63055693).

FIG. 10 is a listing of an amino acid sequence (SEQ ID NO: 10) of an exemplary mussel adhesive polypeptide (GenBank Accession No. BAB 16314.1; GI No. 10641127).

FIG. 11 is a listing of an amino acid sequence (SEQ ID NO: 11) of an exemplary mussel adhesive polypeptide (GenBank Accession No. AAX23968.1; GI No. 60548042).

FIG. 12 is a listing of an amino acid sequence (SEQ ID NO: 12) of an exemplary mussel adhesive polypeptide (GenBank Accession No. AAY29131.1; GI No. 63055728).

### DETAILED DESCRIPTION

This document provides methods and materials for using an adhesive molecule comprising a plurality of DOPA residues to adhere another compound (e.g., a fluorescent molecule, a polymer, an antibiotic or other therapeutic agent, a polypeptide, a nucleic acid, a whitening particle, a remineralization particle, or biological moiety) to biological substrates such as bone, teeth, cartilage, or cells, or non-biological substrates such as plastic, metal, or glass. In some cases, a whitening particle also can be a remineralization particle. For example, this document provides methods and materials for attaching molecules to teeth for providing a whiter appearance, binding minerals to the teeth, breath freshening, delivery of antibacterial agents, or other therapeutic or aesthetic uses.

In some cases, this document provides methods and materials for contacting teeth with an adhesive molecule and one or more whitening particles such as hydroxyapatite particles, to provide the teeth with a whiter appearance and/or to bind remineralization particles to the teeth.

In some cases, this document provides methods and materials for contacting teeth with an adhesive molecule and one or more fluorescence emitting polypeptides (e.g., a blue fluorescent protein (BFP)), and one or more optional whitening particles, to provide the teeth with a whiter appearance that can be maintained after brushing and/or to bind remineralization particles to the teeth.

In some cases, this document provides methods and materials for contacting teeth with an adhesive molecule, one or more non-fluorescent polypeptides, and whitening particles or other compounds. For example, the non-fluorescent polypeptides can serve as scaffolds for attaching multiple adhesive molecules, which in turn can enhance the delivery of another compound (e.g., a whitening particle, remineralization particle, an antibiotic or other therapeutic agent, or a fluorescence-emitting polypeptide) to the teeth and/or mouth. Delivery of the whitening particles can provide the teeth with a whiter appearance that can be maintained after brushing and/or bind remineralization particles the teeth.

The adhesive molecule can include a plurality of DOPA residues and have the ability to interact with or bind to a tooth, a tooth component (e.g., enamel, hydroxyapatite, acquired dental pellicle, cementum, crown, cervix, cementoenamel junction, or apex), or inorganic dental materials such as crowns, caps, braces, or fillings.

Examples of adhesive molecules that can be used as described herein include, without limitation, mussel adhesive polypeptides (e.g., mussel foot proteins 1, 2, 3, 4, 5, 6, or combinations thereof). Mussel adhesive polypeptides can include one or more DOPA residues formed, for example, via enzymatic oxidation of tyrosine residues. For example, 1, 2, 3, 4, 5, 6, 7, 8, 9, 10, 15, 20, 25 percent or more of the total amino acids of a mussel adhesive polypeptide can be DOPA residues. The tyrosine residues of a recombinant polypeptide can be converted to DOPA residues using a tyrosinase (e.g., a mushroom tyrosinase). See Choi et al., Microb Cell Fact., 11:139 (2012).

For example, an adhesive molecule can have at least 80%, 85%, 90%, 95%, 98%, or 99% sequence identity to any one of the amino acid sequences set forth in SEQ ID NOs: 5-29. In some cases, an adhesive molecule can have the amino acid sequence set forth in SEQ ID NOs: 5-29. The percent identity between a particular amino acid sequence and the amino acid sequence set forth in any one of SEQ ID NOs: 5-29 can be determined as follows. First, the amino acid sequences are aligned using the BLAST 2 Sequences (B12seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. B12seq performs a comparison between two amino acid sequences using the BLASTP algorithm. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity is determined by dividing the number of matches by the length of the amino acid sequence in any one of SEQ ID NOs: 5-29, followed by multiplying the resulting value by 100.

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode the amino acid sequences set forth in SEQ ID NOs: 5-29. The degeneracy of the genetic code is well known to the art; i.e., for many amino acids, there is more than one nucleotide triplet that serves as the codon for the amino acid.

A mussel adhesive polypeptide that can be used as described herein can have an amino acid sequence that is naturally occurring in any type of mussel. For example, a mussel adhesive polypeptide that can be used as described herein can have an amino acid sequence that is naturally occurring in Mytilus edulis (common blue mussel), Mytilus byssus, Mytilus galloprovincialis, Mytilus californianus, Mytilus coruscus, Mytilus trossulusor, or Perna viridis (green mussel).

For example, a mussel adhesive polypeptide that can be used as described herein includes, without limitation, mfp-5 from Mytilus galloprovincialis (GenBank Accession No. AAS00463, SEQ ID NO:5), Mytilus edulis (GenBank Accession No. AAL35297.1, SEQ ID NO:6), or Mytilus californianus (GenBank Accession No. ABE01084.1, SEQ ID NO:7); mfp-3 from Mytilus edulis (GenBank Accession No. AAF89290.1, mfp-3 precursor variant 11), Mytilis californianus (GenBank Accession No. AAY29129.1, SEQ ID NO:9) or Mytilus galloprovincialis (GenBank Accession No. BAB16314.1, SEQ ID NO:10); or mfp-1 from Mytilus edulis (GenBank Accession No., AAX23968.1, SEQ ID NO:11), Mytilis californianus (GenBank Accession No. AAY29131.1, SEQ ID NO:12), or Mytilus galloprovincialis (UniProtKB/Swiss-Prot Q27409.1), or a fragment of any of the naturally-occurring mussel adhesive polypeptides.

In some cases, a mfp-1 mussel adhesive polypeptide can include one or more copies of a consensus sequence such as AKPSYPPTYK (SEQ ID NO:13) or PKISYPPTYK (SEQ ID NO:14). For example, a mussel adhesive polypeptide can include 5, 10, 15, 20, 25, 30, 35, 40, 45, 50, 55, 60, 65, 70, or 75 repeats of the consensus sequences set forth in SEQ ID NO:13 or SEQ ID NO:14. In some cases, the proline residues at position 6 and/or 7 are hydroxyproline residues. In some cases, the tyrosine residues at positions 5 and/or 9 are DOPA residues.

In some cases, a mfp-2 mussel adhesive polypeptide can include one or more copies of a consensus sequence such as TDKAYKPNPCVVSKPCKNRGKCIWNGKAYRCKCAYGYGGRHC (SEQ ID NO:15). For example, a mussel adhesive polypeptide can include 2, 4, 5, 6, 7, 8, 9, 10, or 11 repeats of the consensus sequences set forth in SEQ ID NO:15. In some cases, the tyrosine residues at positions 5, 29, 35, and/or 37 of SEQ ID NO:15 are DOPA residues.

In some cases, a mfp-3 mussel adhesive polypeptide can include one or more copies of a consensus sequence such as ADYYGPNYGPPRRYGGGNYNRYNRYGRRYGGYKGWNNGWNRGRRGKYW (SEQ ID NO:16). In some cases, the tyrosine residues at positions 3, 4, 8, 14, 19, 22, 25, 29, 32, and/or 47 of SEQ ID NO:16 are DOPA residues.

In some cases, a mfp-4 mussel adhesive polypeptide can include one or more copies of a consensus sequence such as HVHTHRVLHK (SEQ ID NO:17) or DDHVNDIAQTA (SEQ ID NO:18). For example, a mussel adhesive polypeptide can include 5, 10, 12, 14, 16, 20, 25, 30, 32, 34, 35, or 36 repeats of the consensus sequences set forth in SEQ ID NO:17 or SEQ ID NO:18.

In some cases, a mfp-5 mussel adhesive polypeptide can include one or more copies of a consensus sequence such as SSEEYKGGYYPGNAYHYSGGSYHGSGYHGGYKGKYYGKAKKYYYKYKNS GKYKYLKKARKYHRKGYKYYGGSS (SEQ ID NO:19). In some cases, the tyrosine residues at positions 5, 9, 10, 15, 17, 22, 27, 31, 35, 36, 42, 43, 44, 46, 52, 54, 61, 66, 68, and/or 69 of SEQ ID NO:19 are DOPA residues.

In some cases, a mfp-6 mussel adhesive polypeptide can include one or more copies of a consensus sequence such as GGGNYRGYCSNKGCRSGYIFYDNRGFCKYGSSSYKYDCGNYACLPRNPYGR VKYYCTKKYSCPDDFYYYNNKGYYYYNDKDYGCFNCGSYNGCCLRSGY (SEQ ID NO:20). In some cases, the tyrosine residues at positions 5, 8, 18, 21, 29, 34, 36, 41, 49, 54, 55, 60, 67, 68, 69, 74, 75, 76, 77, 82, 90, and/or 99 of SEQ ID NO:20 are DOPA residues.

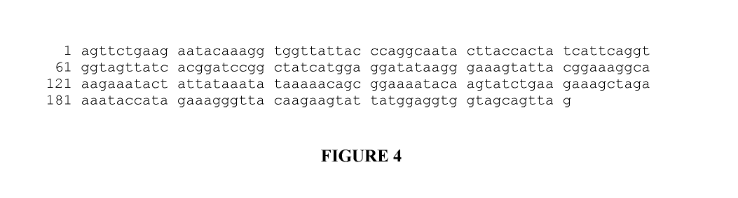

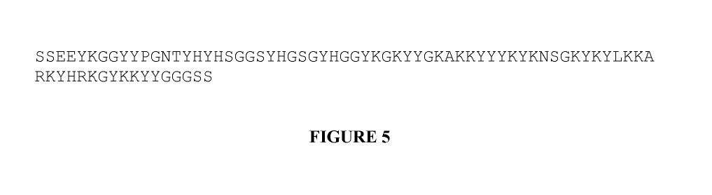

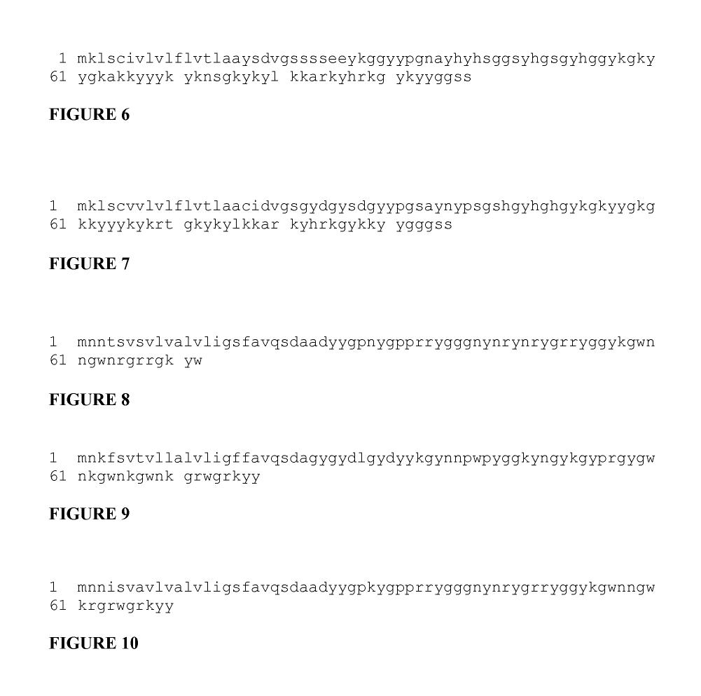

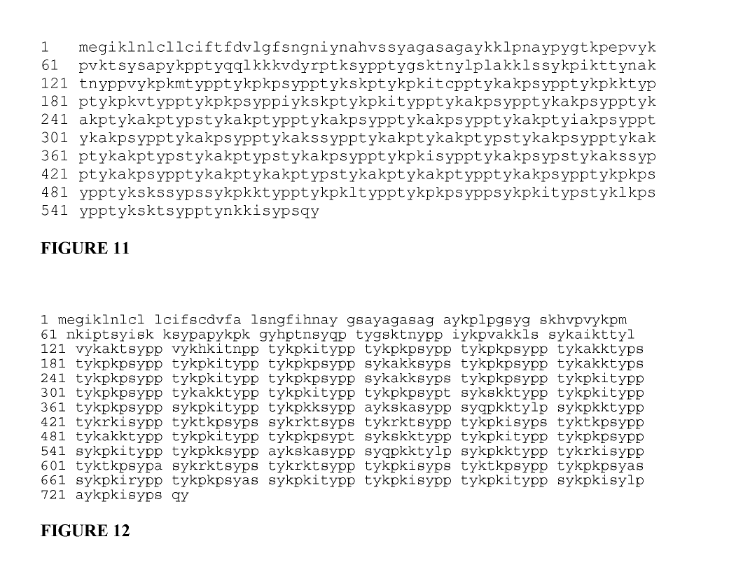

In some cases, a mussel adhesive polypeptide having the amino acid sequence set forth in any one of FIGS. 5-12 (SEQ ID NOs:5-12) or having an amino acid sequence encoded by the nucleotide sequence set forth in FIG. 4 can be used as described herein.

In some cases, a mussel adhesive polypeptide can be a portion of a full-length mussel adhesive polypeptide. For example, a mussel adhesive polypeptide can be used as described herein that includes six repeats of the decapeptide AKPSYPPTYK (SEQ ID NO:13). See, Kitamura et al., J. Polymer Science: Part A: Polymer Chemistry, 37:729-736 (1991).

In some cases, a mussel adhesive polypeptide that can be used as described herein can be a chimeric polypeptide that includes six decapeptide (AKPSYPPTYK; SEQ ID NO:13) repeats of MFP-1 at both the N- and C-termini of MFP-3 (e.g., SEQ ID NO:8). See, Lim, et al., Biomaterials, 31:3715-3722 (2010); and Hwang et al., Biomaterials, 28:3560-3568 (2007). In some cases, the proline residues at position 6 and/or 7 of SEQ ID NO:13 are hydroxyproline residues. In some cases, the tyrosine residues at positions 5 and/or 9 of SEQ ID NO:13 are DOPA residues.

In some cases, a mussel adhesive polypeptide that can be used as described herein can be a chimeric polypeptide that includes six decapeptide (AKPSYPPTYK; SEQ ID NO:13) repeats of MFP-1 at both the N- and C-termini of MFP-5 (SEQ ID NO: 6). See, Lim, et al., Biomaterials, 31:3715-3722 (2010); and Hwang et al., Biomaterials, 28:3560-3568 (2007). In some cases, the proline residues at position 6 and/or 7 of SEQ ID NO:13 are hydroxyproline residues. In some cases, the tyrosine residues at positions 5 and/or 9 of SEQ ID NO:13 are DOPA residues.

Mussel adhesive polypeptides can be extracted from any type of mussel or can be recombinantly produced using polypeptide expression techniques (e.g., heterologous expression techniques using bacterial cells, insect cells, or mammalian cells). Preparations of mussel adhesive polypeptides that are extracted from mussels are commercially available from Cell-Tek (Catalog No. 354240) and ACRO Biosystems (Catalog No. MAP-O4012). In some cases, mussel adhesive polypeptides can be made as described elsewhere (e.g., Kitamura et al., J. Polymer Science: Part A: Polymer Chemistry, 37:729-736 (1991); Lim et al., Biomaterials, 31: 3715-3722 (2010); and Hwang et al., Biomaterials, 28:3560-3568 (2007)). In some cases, standard polypeptide synthesis techniques (e.g., liquid-phase polypeptide synthesis techniques or solid-phase polypeptide synthesis techniques) can be used to produce mussel adhesive polypeptides synthetically.

Other examples of adhesive molecules that can be used as described herein include polymers that include a plurality of DOPA residues. See, for example, the adhesive molecules in Table 1 that contain a plurality of DOPA residues. In some cases, such polymers can have one or more repeats of the consensus sequence XYX₄YX₃YX₃YYX₅YYYXYX₅YXYX₆YX₄YXYYX, where X is lysine, glycine, serine, histidine, or asparagine, and where Y refers to DOPA instead of tyrosine. In cases in which serine is included in the polymer, a phosphoserine residue can be substituted for one or more of the serine residues. For example, in the adhesive molecule of any one of SEQ ID NOs:23-25, a phosphoserine residue can be substituted for one or more of the serine residues. In some cases, an adhesive molecule can be a polypeptide containing a random mixture of DOPA and lysine residues, a random mixture of DOPA, lysine, and glycine residues, or random mixture of DOPA and N5-(2-hydroxyethyl)-L-Glutamine. See, for example, Wang et al., Biomaterials, 28:3456-3468 (2007); and Anderson et al., Advanced Functional Materials, 20:4196-4205 (2010). An adhesive molecule also can be a polyamino acid containing catechols. See, for example, U.S. Pat. No. 6,506,577. Such polymers can range in size from 20 to 1000 amino acids in length, e.g., 20 to 750, 20 to 500, 20 to 350, 20 to 300, 20 to 250, 20 to 200, 20 to 150, 20 to 125, 20 to 100, 30 to 600, 30 to 550, 30 to 500, 30 to 450, 30 to 400, 30 to 350, 30 to 300, 30 to 250, 30 to 200, 30 to 150, 30 to 125, 30 to 100, 50 to 750, 50 to 700, 50 to 650, 50 to 600, 50 to 550, 50 to 500, 50 to 450, 50 to 400, 50 to 350, 50 to 300, 50 to 250, 50 to 200, 50 to 150, 50 to 125, or 50 to 100 amino acids in length.

Suitable polymers can have a peptidic or non-peptidic backbone, and can be synthesized by solid phase or solution phase synthesis. Such synthesis techniques allow a high percentage of DOPA (e.g., greater than 10%, greater than 15%, greater than 20%, greater than 30% of DOPA, or greater than 40% DOPA) to be incorporated into the polymers.

TABLE 1
Adhesive Molecules Containing a Plurality of DOPA
Residues

| | SEQ. |
|---|---|
| Adhesive Molecule, where Y refers to L-DOPA | ID |
| KYKGKGYKGGYKGKYYGKGKKYYYKYKGKGKYKYGKKKGKYKG | 21 |
| KGYKYYG | |
| KYKKKKYKKKYKKKYYKKKKKYYYKYKKKKKYKYKKKKKKYKK | 22 |
| KKYKYYK | |
| SYHGSGYHGGYKGKYYGKAKKYYYKYKNSGKYKYLKKARKYHR | 23 |
| KGYKYYG | |
| SYSSSSYSSSYKSKYYSKSKKYYYKYKSSSKYKYSKKSSKYSS | 24 |
| KSYKYYS | |
| GYSGKNYHGSYKGKYYHKHKKYYYKYKLLHKYKYGKKGNKYGG | 25 |
| KGYKYYH | |
| KYKYKYYKYYYKYKYYYKYKKYYYKYKYKYKYKYYKKKYKYKY | 26 |
| KYYKYYY | |
| KYKGKGGKGGYKGKGYGKGKKGYGKYKGKGKGKYLKKKGKGKG | 27 |
| KGYKGYG | |
| KYKKKKYKKKYKKKYYKKKKKYYYKYKKKKKYKYKKKKKKYKK | 28 |
| KKYKYYK | |
| KKGGYYKGKYKYGKKKKGGYYGGKYYGKKKYGKYYYYYKGKYY | 29 |
| GYKYGKK | |

An adhesive molecule can have a poly(acrylic acid) backbone with a plurality of DOPA residues. For example, an adhesive molecule can have a poly[butadiene-co-(maleic acid)] or poly[ethylene-co-(maleic acid)] backbone with a plurality of DOPA residues, and optionally a plurality of lysine residues, attached as side chains. See, for example, Laulicht et al., Macromol. Biosci., 12: 1555-1565 (2012). Such polymers containing a plurality of DOPA residues are soluble at basic pH values. The addition of lysine residues can increase the solubility at both acid and basic values. An adhesive molecule can have a polymethacrylate backbone with a plurality of DOPA residues, and optionally a plurality of lysine residues, incorporated. See, Kim et al., J. Porous Mater., 20:177-182 (2013). A polymethacrylate polymer containing a plurality of DOPA residues can be soluble at both acid and basic pH values.

In some cases, an adhesive molecule can be a DOPA containing polypeptide or a poly(dopamine) polymer. See, for example, Fuller et al., Biopolymers, 17:2939-2943 (1998); and Lee et al., Adv. Mater., 21:431-434 (2009). A poly(dopamine) polymer can be prepared, for example, by in situ polymerization.

In some cases, an adhesive molecule can be a polyethylene glycol terminated with DOPA. See, for example, Dalsin et al., J. Am. Chem. Soc., 125:4253-4258 (2003).

In some cases, the adhesive molecule and other molecule (e.g., polypeptide, nucleic acid, fluorescent moiety, antibiotic or other drug) can be applied sequentially to a substrate (e.g., a biological or non-biological substrate). For example, an adhesive molecule and one or more polypeptides (e.g., fluorescent or non-fluorescent polypeptides) can be applied sequentially, i.e., the adhesive molecule can be applied to teeth (under dry conditions, under wet conditions, or under conditions typically found in the mouth, e.g., saliva on the teeth) and then the one or more polypeptides can be applied.

In some cases, the adhesive molecule and other molecule (e.g., polypeptide, nucleic acid, fluorescent moiety, antibiotic or other drug) can be applied together, e.g., the adhesive molecule and other molecule are attached to each other (e.g., conjugated to each other). For example, an adhesive molecule and fluorescence emitting polypeptide can be conjugated to each other or an adhesive molecule and a non-fluorescent polypeptide can be conjugated to each other. The fluorescence emitting polypeptides are applied to teeth (under dry conditions, under wet conditions, or under conditions typically found in the mouth, e.g., saliva on the teeth) such that fluorescence is emitted from the teeth. In some cases, the non-fluorescent polypeptides and adhesive molecule are conjugated to each other and applied to teeth under dry conditions, under wet conditions, or under conditions typically found in the mouth, e.g., saliva on the teeth, such that another compound (e.g., a whitening particle or therapeutic agent) can be delivered to the teeth. The non-fluorescent polypeptides can be, for example, collagen or albumin.

Any appropriate fluorescence emitting polypeptide can be used. For example, when the desire is to have whiter appearing teeth, a polypeptide that emits blue fluorescence can be applied to a person's teeth. Such blue fluorescence can have an emission wavelength between about 440 nm and about 500 nm (e.g., between about 450 nm and about 500 nm, between about 460 nm and about 500 nm, between about 470 nm and about 500 nm, between about 480 nm and about 500 nm, between about 440 nm and about 490 nm, between about 440 nm and about 480 nm, between about 440 nm and about 470 nm, between about 440 nm and about 460 nm, between about 450 nm and about 490 nm, or between about 460 nm and about 480 nm). In some cases, a fluorescence emitting polypeptide that emits fluorescence at an emission wavelength of between about 420 nm and about 450 nm, between about 430 nm and about 450 nm, between about 440 nm and about 450 nm, between about 420 nm and about 440 nm, or between about 485 nm and about 505 nm can be applied to teeth as described herein.

When the desire is to have teeth of a different color, a polypeptide that emits fluorescence in the red, green, or yellow spectrum can be applied to the person's teeth. Red fluorescence can have an emission wavelength between about 555 nm and about 655 nm (e.g., between about 565 nm and about 645 nm, between about 575 nm and about 635 nm, or between about 585 nm and about 625 nm). Green fluorescence can have an emission wavelength between about 500 nm and about 525 nm (e.g., between about 505 nm and about 520 nm or between about 510 nm and about 515 nm). Yellow fluorescence can have a wavelength between about 525 nm and about 555 nm (e.g., between about 530 nm and about 550 nm or 535 nm and about 545 nm). In some cases, a combination of different fluorescence emitting polypeptides can be applied to a person's teeth. For example, a combination of BFP polypeptides and red fluorescent protein (RFP) polypeptides can be applied to a person's teeth. In some cases, a combination of RFP polypeptides and green fluorescent protein (GFP) polypeptides can be applied to a person's teeth.

Any appropriate BFP polypeptide can be used as described herein. Examples of BFP polypeptides that can be used as described herein include, without limitation, EBFP (e.g., an EBFP having an emission max of 460 nm), fluorescent protein SBFP1 (GenBank® Accession No. ABM97856; GI No. 124264536), fluorescent protein SBFP2 (GenBank® Accession No. ABM97857, GI No. 124264538), EBFP2 (GenBank® Accession No. EF517318, GI No. 145666498), Azurite (Mena et al., Nature Biotechnology, 24:1569-1571 (2006)), mKalama1 (GenBank® Accession No. EF517317, GI No. 145666496), zinc finger protein 383 (GenBank® Accession No. EDU39924.1, GI No. 187972425), SEQ ID NO:445 set forth in U.S. Pat. No. 7,166,424 (GenBank® Accession No. ABN30727.1; GI No. 125148618), soluble-modified blue fluorescent protein (smBFP) (GenBank® Accession No. U70497.1; GI No.1619752), polypeptides having the sequence set forth in GenBank® Accession No. CAE00365.1 (GI No. 32260521), polypeptides having the sequence set forth in GenBank® Accession No. CAE00361.1 (GI No. 32260509), polypeptides having the sequence set forth in GenBank® Accession No. CAE00361.1 (GI No. 32260509), ECFP polypeptides (GenBank® Accession No. AC048275.1; GI No. 226331138), Cerulean polypeptides (GenBank® Accession No. ADE48834.1; GI No. 293612838), Fluorescent Protein Cypet polypeptides (GenBank® Accession No. 3GEX_A; GI No. 290789997), MiCy polypeptides (GenBank® Accession No. ADE48830.1; GI No. 293612833), and mTFP1 fluorescent protein polypeptides (GenBank® Accession No. AC048263.1; GI No. 226320339). In some cases, a BFP polypeptide set forth in U.S. Patent Application Publication No. 2010/0062460 can be used as described herein.

Any appropriate RFP polypeptide and GFP polypeptide can be used as described herein. Examples of RFP polypeptides that can be used as described herein include, without limitation, soluble-modified red-shifted green fluorescent protein (smRSGFP) polypeptides (GenBank® Accession No. U70496.1; GI No.1619750), red fluorescent protein polypeptides having the sequence set forth in GenBank® Accession No. AAG16224.1 (GI No. 10304307); AB038175.1 (GI No.133753343); or AAU06852.1 (GI No. 51593130), Orange-Emitting Gfp-Like Protein polypeptides (GenBank® Accession No. 2ZMW_D; GI No. 209870302), mOrange fluorescent protein polypeptides (GenBank® Accession No. AC048285.1; GI No. 226331152), NLS-dTomato polypeptides (GenBank® Accession No. ADC42843.1; GI No. 288188779), red fluorescent protein tdTomato polypeptides (GenBank® Accession No. ACQ43939.1; GI No. 228484713), DsRed polypeptides (GenBank® Accession No. BAE53441.1; GI No. 83016748), DsRed2 polypeptides (GenBank® Accession No. AAV73970.1; GI No. 56119204), DsRed-Express polypeptides (GenBank® Accession No. ACU30027.1; GI No. 255689290), DsRed-Monomer polypeptides (GenBank® Accession No. ACF35425.1; GI No. 194245628), monomeric orange-red fluorescent protein polypeptides (GenBank® Accession No. AAV52170.1; GI No. 55420625), monomeric orange-red fluorescent protein polypeptide (GenBank® Accession No. AAV52166.1; GI No. 55420617), mCherry polypeptides (GenBank® Accession No. ACY24904.1; GI No. 262089840), polypeptides having the amino acid sequence of SEQ ID NO:3 set forth in U.S. Pat. No. 7,393,923 (GenBank® Accession No. ACH06540.1; GI No. 197013979), and polypeptides having the amino acid sequence of SEQ ID NO:5 set forth in U.S. Pat. No. 7,393,923 (GenBank® Accession No. ACH06541.1; GI No. 197013980).

Examples of GFP polypeptides that can be used as described herein include, without limitation, soluble-modified green fluorescent protein (smGFP) polypeptides (GenBank® Accession No. U70495.1; GI No.1619748), modified green fluorescent protein GFP-ER (mfgp4-ER) polypeptides (GenBank® Accession No. U87625.1; GI No. 1842446), GFP polypeptides (GenBank® Accession No. ACJ06700.1, GI No. 210076685), enhanced GFP polypeptides (GenBank® Accession No. ACV20892.1; GI No. 256708579), turboGFP polypeptides (GenBank® Accession No. ADD23343.1; GI No. 290131407), VisGreen GFP polypeptides (GenBank® Accession No. ABR26680.1; GI No. 149393496), and Azami-Green polypeptides (GenBank® Accession No. BAD52001.1; GI No. 52839539).

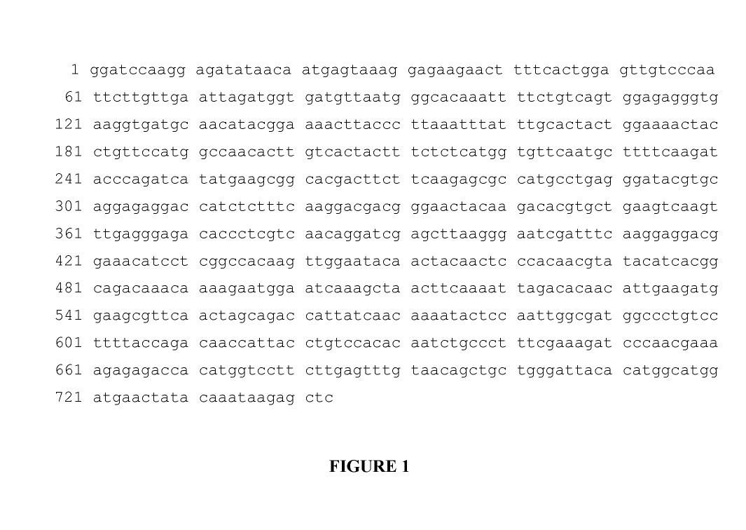

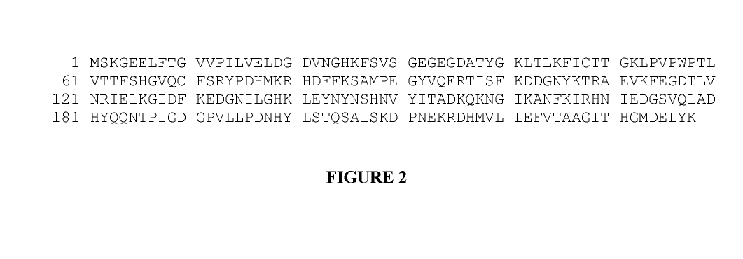

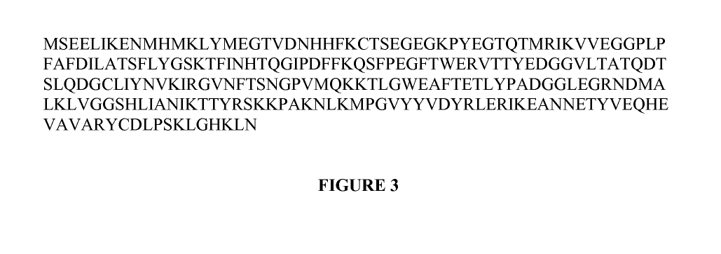

In some cases, a fluorescence emitting polypeptide such as those described by Subach et al. (Chem. Biol., 15:1116-1124 (2008)) can be used as described herein. See, also, GenBank® Accession No. 3M24_A (GI No. 296863586), GenBank® Accession No. 3M24 B (GI:296863587), GenBank® Accession No. 3M24_C (GI:296863588), and GenBank® Accession No. 3M24_D (GI:296863589). Additional examples of fluorescence emitting polypeptides that can be used as described herein include, without limitation, those described elsewhere (Alieva et al. PLoS ONE, 3(7):e2680 (2008) and Chudafov et al., Physiol. Rev., 90:1103-1163 (2010)). See, e.g., Table 1 of the Alieva et al. reference and FIGS. 5, 10, 12, and 14 of the Chudafov et al. reference. In some cases, a coral fluorescence emitting polypeptide can be used as described herein. In some cases, a fluorescence emitting polypeptide having the amino acid sequence set forth in FIG. 2 or 3 or having an amino acid sequence encoded by the sequence set forth in FIG. 1 can be used as described herein.

Any appropriate method can be used to make a polypeptide (e.g., a fluorescence emitting polypeptide or a non-fluorescent polypeptide). For example, polypeptide expression techniques (e.g., heterologous expression techniques using bacterial cells, insect cells, or mammalian cells) can be used to make a polypeptide. In some cases, fluorescence emitting polypeptides such as BFP polypeptides can be made as described elsewhere (Yakhnin et al., Protein Expr. Purif., 14:382-386 (1998) and Jain et al., J. Chromatography A, 1035:83-86 (2004)). In some cases, standard polypeptide synthesis techniques (e.g., liquid-phase polypeptide synthesis techniques or solid-phase polypeptide synthesis techniques) can be used to produce polypeptides (e.g., fluorescence emitting polypeptides or a non-fluorescent polypeptides) synthetically.

A polypeptide can be covalently or non-covalently attached to an adhesive molecule such as a mussel adhesive polypeptide or polymer containing a plurality of DOPA residues. Any appropriate method can be used to covalently or non-covalently attach a fluorescence emitting polypeptide or a non-fluorescent polypeptide to an adhesive molecule (e.g., a polypeptide or polymer) having the ability to interact with or bind to a tooth or a tooth component. For example, a fluorescence emitting polypeptide such as a BFP polypeptide or a non-fluorescent polypeptide such as collagen or albumin can be chemically conjugated to an adhesive molecule such as a mussel adhesive polypeptide or polymer via one or more coordinate covalent bonds, covalent bonds, disulfide bonds, high energy bonds, hydrogen bonds, ionic bonds, or peptide bonds. In some cases, a fluorescence emitting polypeptide or a non-fluorescent polypeptide can be chemically conjugated to an amine group present on a polypeptide having the ability to interact with or bind to a tooth or a tooth component (e.g., a mussel adhesive polypeptide or other polypeptide with a plurality of DOPA residues). Such an amine group can be located at the N-terminus of the polypeptide, the C-terminus of the polypeptide, or in between the N- and C-termini of the polypeptide.

In some cases, the polypeptides to be conjugated can be activated prior to conjugation. For example, a polypeptide (e.g., an adhesive molecule, a fluorescence emitting polypeptide, or a non-fluorescent polypeptide) can be activated by incorporation of a reactive thiol group (e.g., by reaction with 2-iminothiolane such as a Traut's reagent, or reaction with a polyethylene glycol polymer containing a N-Succinimidyl 3-(2-pyridyldithio)-propionate (SPDP) moiety on one end and a N-hydroxysuccinimide ester on the other end, and cleavage of the SPDP moiety with a reducing agent such as dithiothreitol (DTT) to activate the thiol). For example, a mussel adhesive polypeptide can be thiolated by reaction with 2-iminothiolane (e.g., a Traut's reagent) as described elsewhere (McCall et al., Bioconjugate Chem., 1:222-226 (1990)). The reaction conditions can be varied to maximize the yield of molecules activated with one or two thiols to decrease the possibility that conjugation may interfere with teeth binding. The degree of thiol incorporation can be measured using a sensitive fluorescence assay as described elsewhere (Lacy et al., Analytical Biochemistry, 382:66-68 (2008)).

An adhesive molecule or a polypeptide (e.g., a fluorescence emitting polypeptide or a non-fluorescent polypeptide) can be substituted with one or more maleimide groups via reaction of the polypeptide's amines with a bifunctional reagent containing a maleimide group and a reactive N-hydroxysuccinimide ester (e.g., a polyethylene glycol polymer containing a maleimide group on one end and a reactive N-hydroxysuccinimide ester on the other). The maleimide substituted fluorescence emitting polypeptide or non-fluorescent polypeptide can then be conjugated to the thiol groups of the polypeptide having the ability to interact with or bind to a tooth or a tooth component. In some cases, a maleimide substituted adhesive molecule can be conjugated to the thio groups of a polypeptide. The degree to which the polypeptide (e.g., a fluorescence emitting polypeptide or a non-fluorescent polypeptide) or adhesive molecule is substituted with maleimide groups can be varied as described elsewhere (Singh, Bioconjugate Chem., 5:348-351 (1994)).

Additional examples of conjugation methods that can be used to conjugate a fluorescence emitting or non-fluorescent polypeptide to a molecule (e.g., a polypeptide) having the ability to interact with or bind to a tooth or a tooth component include, without limitation, those described in elsewhere (e.g., Hermanson, G. T. Bioconjugate Techniques, Second Edition, 2008, Elsevier). See, e.g., Part I, Section 4 and Part II, Section 5.

In some cases, the fluorescent signal that is obtained using the methods and materials provided herein can be enhanced by linking multiple fluorescence emitting polypeptides to a single molecule (e.g., a polypeptide) having the ability to interact with or bind to a tooth or a tooth component. Because of steric effects, this amplification can be effectively accomplished by first preparing a polymer containing multiple fluorescence emitting polypeptides and then linking this polymer to an adhesive molecule (e.g., a mussel adhesive polypeptide) having the ability to interact with or bind to a tooth or a tooth component. In some cases, a polymer can be formed to have multiple fluorescence emitting polypeptides linked to a polypeptide such as a casein polypeptide, and this polymer can be applied with the adhesive molecule. Examples of methods (e.g., polymerization methods) that can be used to form polymers containing multiple fluorescence emitting polypeptides include, without limitation, those described elsewhere (e.g., Hermanson, G. T. Bioconjugate Techniques, Second Edition, 2008, Elsevier). See, e.g., Part II, Section 25. See also U.S. Patent Publication No. 2010/0203533; U.S. Patent Publication No. 2013/0022555, including the sections describing conjugations to casein polypeptides; U.S. Pat. No. 4,657,853; and Hoshino et al., J. Biochem. 102:785-791 (1987).

Conjugating an adhesive molecule activated with multiple maleimide groups to a fluorescence emitting or a non-fluorescent polypeptide containing multiple thiol groups can produce monomeric adhesive molecule—polypeptide conjugates as well as oligomers that contain different number of adhesive molecules and polypeptides. The molecular weight distribution of such covalently linked oligomers can be determined by gel electrophoresis under denaturing conditions (SDS-PAGE). The molecular weight distribution of the products depends on a number of factors such as the degree to which the adhesive molecule and polypeptide are activated, the pH of the conjugation reaction (e.g., about pH 5 to about pH 7, e.g., pH 5 to 6), and the ratio of adhesive molecule: polypeptide in the conjugation reaction. In some embodiments, adhesive molecule:fluorescent molecule oliogmers with molecular weights greater than approximately 200,000 Daltons can, after complexation with hydroxyapatite, show a decreased ability to whiten teeth.

In addition to the molecular weight distribution of oligomers that contain different number of adhesive molecules and polypeptides, such oligomers can associate via non-covalent interactions in a process called aggregation. The aggregation state of the oligomers can be determined by size exclusion chromatography analysis under native conditions. In some embodiments, aggregation can impact whitening. Accordingly, to minimize aggregation, factors such as the pH of the conjugation reaction, storage pH, and ionic strength can be modulated as well as the concentration of the adhesive molecule and polypeptide (e.g., fluorescence-emitting or non-fluorescent polypeptide) and the degree of activation. Typically, aggregation decreases when the pH of the conjugation reaction is between about pH 5 and about pH 6, and when the conjugation reaction is stored at a pH of about 5 to about pH 6.0. Aggregation also typically is minimized when the ionic strength is >5 mM, e.g., 50 mM. Lower concentration, e.g., <1 mg/mL, <0.5 mg/mL, <0.2 mg/mL, or <0.1 mg/mL of the adhesive molecule and fluorescence-emitting polypeptide or non-fluorescent polypeptide also can minimize aggregation.

Conjugation reactions between adhesive molecules and polypeptides (e.g., fluorescence-emitting polypeptides or non-fluorescent polypeptides) also are performed such that the resulting conjugates are stable under conditions typically found in the mouth (e.g., for a period of one to seven days, or for one or more weeks such as two, three, four, or more weeks) and do not result in oxidation or discoloration.

In some cases, a fluorescence emitting polypeptide can be produced as a fusion or chimeric polypeptide with a polypeptide having the ability to interact with or bind to a tooth or a tooth component such that the fusion or chimeric polypeptide has the ability to both emit fluorescence and to interact with or bind to a tooth, a tooth component, or inorganic dental material. For example, heterologous polypeptide expression techniques or synthetic polypeptide synthesis techniques can be used to produce a single polypeptide chain having an amino acid sequence of a full-length fluorescence emitting polypeptide or fragment thereof and an amino acid sequence of an adhesive molecule having the ability to interact with or bind to a tooth or a tooth component (e.g., an adhesive molecule such as a mussel adhesive polypeptide or a polymer containing a plurality of DOPA residues). For example, a chimeric polypeptide can include a full length fluorescence emitting polypeptide or fragment thereof that is at least about 90 percent identical to the full length fluorescence emitting polypeptide, and an adhesive molecule (e.g., a full length mussel adhesive polypeptide or fragment thereof that is at least about 80 percent identical to the full length mussel adhesive polypeptide or a polymer containing a plurality of DOPA residues such as the polymers set forth in Table 1).

In some cases, the single polypeptide chain can have (a) an amino acid sequence of a fluorescence emitting polypeptide followed by an amino acid sequence of a polypeptide having the ability to interact with or bind to a tooth or a tooth component or (b) an amino acid sequence of a polypeptide having the ability to interact with or bind to a tooth or a tooth component followed by an amino acid sequence of a fluorescence emitting polypeptide. In some cases, the single polypeptide chain can have one or more (e.g., one, two, three, four, or five) amino acid sequences with each encoding a fluorescence emitting polypeptide and one or more (e.g., one, two, three, four, or five) amino acid sequences with each encoding a polypeptide having the ability to interact with or bind to a tooth or a tooth component.

In some cases, a fusion or chimeric polypeptide provided herein can include other amino acid sequences (e.g., spacers or binding residues). For example, a fusion or chimeric polypeptide having an amino acid sequence of a fluorescence emitting polypeptide and an amino acid sequence of a polypeptide having the ability to interact with or bind to a tooth or a tooth component can include one or more additional amino acid residues such as glycine, lysine, alanine, arginine, asparagine, aspartic acid, cysteine, glutamine acid, glutamine, isoleucine, leucine, methionine, phenylalanine, threonine, tryptophan, proline, histidine, valine serine, tyrosine, ornithine, taurine, pyrolysine, or seleocysteine residues, or amino acid derivatives (e.g., 5-hydroxytryptophan, L-dihydroxyphenylalanine, or α-difluoromethylornithine). Such additional amino acid residues can be designed to be spacers (e.g., a string of five or more glycine residues) or can be designed to allow polypeptides or other molecules to be chemically conjugated to the fusion or chimeric polypeptide. For example, a fusion or chimeric polypeptide having an amino acid sequence of a fluorescence emitting polypeptide and an amino acid sequence of a polypeptide having the ability to interact with or bind to a tooth or a tooth component can include one, two, three, four, five, or more additional lysine residues such that one or more polypeptides having the ability to interact with or bind to a tooth or a tooth component (e.g., mussel adhesive polypeptides) can be chemically conjugated to the fusion or chimeric polypeptide.

The methods described herein can include using the adhesive molecule and a non-fluorescent polypeptide to apply whitening particles and/or remineralization particles composed, for example, of hydroxyapatite, substituted hydroxyapatite, amorphous calcium phosphate, or fluoride, calcium, magnesium, phosphate, iron, or tin ions, and any salt forms thereof (e.g., sodium hexametaphosphate, magnesium chloride, ferrous sulfate) to teeth to provide the teeth with a whiter appearance and/or to bind remineralization particles to the teeth.

The methods described herein can include applying whitening particles or remineralization particles to teeth instead of, or in addition to, the fluorescence emitting polypeptide, to provide the teeth with a whiter appearance and/or bind remineralization particles to the teeth.

Whitening particles can be nanoparticles or microparticles, or aggregates of nanoparticles or microparticles, and can range in size from 1 nanometer (nm) to 100 micrometers (μm) in size such as 1 nm to 50 μm, 1 nm to 20 μm, 5 nm to 20 μm, 10 nm to 20 μm, 1 nm to 10 μm, 5 nm to 10 μm, 10 nm to 10 μm, 1 nm to 1 μm, 5 nm to 1 μm, 10 nm to 1 μm, 100 nm to 1 μm, 1 nm to 500 nm, 1 nm to 250 nm, 1 nm to 125 nm, 1 nm to 100 nm, 1 nm to 75 nm, 1 nm to 50 nm, 5 nm to 500 nm, 5 nm to 250 nm, 5 nm to 125 nm, 5 nm to 100 nm, 5 nm to 75 nm, 5 nm to 50 nm, 5 nm to 20 nm, 10 nm to 150 nm, 10 nm to 125 nm, 10 nm to 100 nm, 10 nm to 75 nm, 10 nm to 50 nm, or 50 nm to 150 nm, or 50 nm to 125 nm in size. Whitening particles can be composed of hydroxyapatite or titanium dioxide. Other useful whitening particles can be composed of silicon dioxide, zirconium silicate, calcium phosphate, or zinc oxide. See, e.g., Photochem. Photobiol. Sci., 9, 495-509 (2010); and U.S. Pat. No. 6,004,567. In some cases, whitening particles composed of hydroxyapatite also are remineralization particles.

In some cases, adhesive molecules and whitening particles (or other compound such as a remineralization particle) can be applied sequentially, i.e., the adhesive molecule can be applied to teeth and then the whitening particles (or other compound) can be applied. In some cases, the adhesive molecule and whitening particles (or other compound) can be applied at the same time. In some cases, the adhesive molecule, the non-fluorescent polypeptide, and the whitening particles can be applied sequentially. In some cases, the adhesive molecule, the fluorescence emitting polypeptide, and the whitening particles can be applied sequentially. In some cases, the adhesive molecule, the non-fluorescent polypeptide, and the whitening particles can be applied at the same time. In some cases, the adhesive molecule, the fluorescence emitting polypeptide, and the whitening particles can be applied at the same time. For example, the whitening particles can be bound to a conjugate containing an adhesive molecule and a fluorescent emitting polypeptide, and the complex containing the whitening particles and conjugate can be applied to the teeth. In some cases, using the whitening particles in combination with the fluorescence emitting polypeptides can help even out the whiter appearance of the teeth.

In some cases in which titanium dioxide or other particles are used, the particles can be coated with one or more serum proteins such as albumin or immunoglobulin (e.g., by incubating the particles with serum) that bind non-specifically to titanium dioxide, and the coated particles can be applied in combination with an adhesive molecule (e.g., polymer containing a plurality of DOPA residues). In some cases, a serum protein can be activated and chemically conjugated to an adhesive molecule/fluorescence emitting polypeptide conjugate, and then the conjugate containing the serum protein, adhesive molecule, and fluorescence emitting polypeptide can be bound to whitening particles such as titanium dioxide particles.

A composition provided herein containing an adhesive molecule and one or more other molecules (e.g., a composition containing an adhesive molecule attached to an antibiotic or other therapeutic molecule such as insulin or human growth hormone) can be administered to a subject to deliver the other molecule(s) to particular locations within a body. For example, a composition containing an adhesive molecule and an antibiotic can be formulated as a gel, mouthrinse, mouthwash, or deodorant. In some cases, the composition can include one or more pharmaceutical excipients.

A composition provided herein containing an adhesive molecule and a fluorescence emitting polypeptide (e.g., a composition containing a fluorescence emitting polypeptide/mussel adhesive polypeptide conjugate, or a fluorescence emitting polypeptide/mussel adhesive polypeptide fusion polypeptide) can be applied to teeth under dry or wet conditions to alter the appearance of the teeth. In some cases, the composition can be applied under typical conditions found in the mouth (e.g., the presence of saliva). A composition provided herein containing an adhesive molecule and whitening particles (e.g., a composition containing an adhesive molecule and titanium dioxide or hydroxyapatite nanoparticles) can be applied to teeth under dry or wet conditions to alter the appearance of the teeth.

A composition provided herein containing an adhesive molecule, a non-fluorescent polypeptide, and other compound (e.g., whitening particles, remineralization particle, polypeptide, nucleic acid, therapeutic agent, or combinations thereof) can be applied to teeth under dry or wet conditions to alter the appearance of the teeth. For example, a composition containing hydroxyapatite particles and a non-fluorescent polypeptide/mussel adhesive polypeptide conjugate or a non-fluorescent polypeptide/adhesive molecule comprising a plurality of DOPA residues can be applied to teeth under dry or wet conditions to alter the appearance of the teeth and/or bind remineralization particles. In some cases, the composition can be applied under typical conditions found in the mouth (e.g., the presence of saliva).

A composition provided herein containing an adhesive molecule, a fluorescence emitting polypeptide, and whitening particles can be applied to teeth under dry or wet conditions to alter the appearance of the teeth. For example, a composition containing hydroxyapatite particles and a fluorescence emitting polypeptide/mussel adhesive polypeptide conjugate, a fluorescence emitting polypeptide/adhesive molecule comprising a plurality of DOPA residues, or a fluorescence emitting polypeptide/mussel adhesive polypeptide fusion polypeptide can be applied to teeth under dry or wet conditions to alter the appearance of the teeth. For example, a composition containing titanium dioxide particles that have been incubated in serum to coat the titanium dioxide with serum proteins and a fluorescence emitting polypeptide/adhesive molecule conjugate, or a fluorescence emitting polypeptide/adhesive molecule fusion polypeptide can be applied to teeth under dry or wet conditions to alter the appearance of the teeth. In some cases, the composition can be applied under typical conditions found in the mouth (e.g., the presence of saliva).

Any appropriate method can be used to deliver a composition provided herein to teeth. For example, a composition provided herein can be incorporated into a tooth paste, a mouth wash, a mouth rinse, an ingestable substance such as a drink or a food product, gum (e.g., chewing gum), gels (an application gel), powders, or creams. In some cases, the composition can include one or more pharmaceutical excipients. For example, a toothpaste containing an adhesive molecule, a polypeptide, and whitening particles or a toothpaste containing another composition described herein can include one or more thickeners (e g , mineral colloids or polyethylene glycol (PEG)), buffers, surfactants, fluorides, flavorings (e.g., peppermint, spearmint, wintergreen, or bubble gum), sugar alcohols (e.g., sorbitol, glycerol, or xylitol), sensitivity reducers (e.g., potassium nitrate) and/or anti-bacterial agents (e.g., Triclosan or zinc chloride) that do not interfere with whitening of teeth and/or binding of remineralization particles to teeth.

In some cases, an effective amount of the adhesive molecule and polypeptide (e.g., fluorescence-emitting polypeptide or non-fluorescent polypeptide) and/or whitening particles can be delivered to teeth such that the appearance of the teeth is altered.

In some cases, an effective amount of a composition provided herein can be delivered to teeth such that the appearance of the teeth is altered (e.g., the appearance of the teeth becomes whiter and/or remineralization particles are bound to the teeth). An effective amount of adhesive molecules, polypeptides, and/or whitening particles or other compound, or a composition provided herein can be any amount that alters the appearance of teeth (e.g., the appearance of the teeth becomes whiter and/or the remineralization particles are bound to the teeth) without inducing significant toxicity. For example, a composition provided herein can be incorporated into a tooth paste, mouth rinse, or gel product in an amount that results in between about 0.0001 mg and about 100 mg (e.g., between about 0.001 mg and about 100 mg, between about 0.01 mg and about 100 mg, between about 0.1 mg and about 100 mg, between about 0.5 mg and about 100 mg, between about 0.5 mg and about 50 mg, between about 0.5 mg and about 25 mg, between about 1 mg and about 100 mg, between about 1 mg and about 50 mg, or between about 1 mg and about 25 mg) of polypeptide (e.g., fluorescence-emitting polypeptide or non-fluorescent polypeptide) per gram of tooth paste. It will be appreciated that the amount can be higher for certain formulations, e.g., mouthwash.

In some cases, a composition provided herein can be applied to teeth for a period of time prior to washing, swallowing, or removal such that the appearance of the teeth is altered (e.g., the appearance of the teeth becomes whiter). For example, a tooth paste or application configured to include an adhesive molecule and a polypeptide and/or whitening particles or other compound as described herein can be applied (e.g., directly applied) to teeth and remain in contact with those teeth, without rinsing, for between 30 seconds and 10 minutes (e.g., between 30 seconds and 5 minutes, between 30 seconds and 2 5 minutes, between 30 seconds and two minutes, between 1 minute and 10 minutes, between 2 minutes and 10 minutes, or between one minute and 5 minutes). In some cases such as with a mouth wash, mouth rinse, application gel or ingested substance, the composition is allowed to be in contact with the teeth for a period of time for the composition to saturate the teeth or tooth component. The compositions described herein can be applied to teeth under wet or dry conditions.

In some cases, a person's teeth can be prepared prior to delivering a composition provided herein. For example, a person's teeth can be washed, brushed (e.g., with an ultrasonic toothbrush), or polished (e.g., polished with pumice) prior to delivering a composition provided herein. Brushing, for example, with an ultrasonic toothbrush can help disrupt the dental pellicle and increase whitening. In some cases, the surface of the tooth or teeth to be treated can be treated with one or more agents capable of exposing calcium phosphate binding sites. For example, teeth to be treated with a composition provided herein can be contacted with EDTA or phosphoric acid to expose calcium phosphate binding sites present on the teeth. In the case of phosphoric acid treatment, only the tooth enamel can be exposed to the acid to prevent or reduce the risk of soft tissue damage.

In some cases, a two or more step process can be used to apply the adhesive molecule and polypeptide (e.g., non-fluorescent polypeptide or fluorescence emitting polypeptide) or other compound to teeth. For example, a composition containing an adhesive molecule (e.g., mussel adhesive polypeptide or polymer containing a plurality of DOPA residues) having the ability to interact with or bind to a tooth, a tooth component, or inorganic dental material can be delivered to the teeth to be treated as one step followed by a step of delivering, for example, a fluorescence emitting polypeptide, non-fluorescent polypeptide, whitening particle, or remineralization particle, having the ability to interact with or bind to the adhesive molecule. In some case, these two steps can be performed at the same time using a single composition that contains the molecule separate from the fluorescence emitting polypeptide or using separate compositions where one composition contains the adhesive molecule and another composition contains the fluorescence emitting polypeptide.

In some cases, an assay can be performed to confirm that a composition provided herein or a component of a composition provided herein (e.g., a mussel adhesive polypeptide or polymer containing a plurality of DOPA residues) has binding affinity for teeth, a tooth component, or inorganic dental material. For example, a material to be tested can be incubated with a hydroxyapatite (HA) matrix and the amount of material in solution after HA binding can be compared with the initial concentration to determine, by difference, the amount of bound material. See, e.g., Raj et al., J. Biol. Chem., 267:5968-5976 (1992). In some cases, the HA bound material can be directly measured after dissolving the HA matrix with EDTA (Lamkin et al., J. Dent. Res., 75:803-808 (1996)). In the case of a polypeptide material, the polypeptide concentration in solution can be measured using a bicinchoninic acid assay and/or an ortho-phthalaldehyde amine assay. Binding constants can be determined using the Langmuir Model (Bouropoulos and Moradian-Oldak, Calcif. Tissue Int., 72:599-603 (2003)). In some case, an assay can be performed with an HA matrix that was pre-incubated with human saliva to coat the HA with proteins as described elsewhere (Lamkin et al., J. Dent. Res., 75:803-808 (1996)). In such cases, unbound saliva proteins can be removed by washing since their presence may interfere with the polypeptide concentration determinations.

Any appropriate method can be used to assess the affinity of a composition provided herein for teeth or an HA matrix. For example, bound and unbound compositions can be quantified by measuring the fluorescence of the fluorescence emitting polypeptide of the composition. The ability to utilize fluorescence for quantification can allow for one to measure the binding of the composition to HA in the presence of human saliva. In some cases, a composition provided herein can be assessed for the ability to bind in vitro to human teeth. The teeth can be subjected to different degrees of cleaning, such as brushing or polishing with pumice The teeth can then be treated with human saliva to form the acquired dental pellicle and incubated with a composition provided herein in the presence and absence of saliva. The binding to teeth can be determined both by measuring the bound and unbound fluorescence. The extraction of bound fluorescence emitting polypeptides from teeth can be completed using gentle procedures. For example, teeth can be swabbed with filter paper collection strips, and the polypeptides can be extracted from the paper under mild conditions as described elsewhere (Siqueira and Oppenheim, Archives of Oral Biology, 54:437-444 (2009)). In some cases, the teeth can be analyzed by fluorescence microscopy to assess the relative amount of fluorescence emitting polypeptide bound under different conditions.

Any appropriate method can be used to assess a composition provided herein for the ability to alter the appearance of teeth and/or bind remineralization particles to the teeth. For example, visual inspection techniques can be used to determine whether or not a composition provided herein can alter the appearance of teeth. Such visual inspection techniques can include using shade guides for comparison as described elsewhere (Paravina et al., J. Esthet. Restor. Dent., 19:276-283 (2007)). In some cases, the ability of a composition provided herein to alter the appearance of teeth (e.g., to make teeth appear whiter) can be measured using reflectance spectrophotometry. In such cases, the teeth can be illuminated with a white light source and analyzed as to the amount of light absorbed at different wavelengths by reflectance spectrophotometry (colorimetry). These measurements can then be repeated with the UV light filtered from the light source. The difference in the reflectance values obtained with the inclusion and exclusion of UV light is the UV fluorescence spectrum of the tooth surface (see, e.g., Park et al., M. Dental Materials, 23:731-735 (2007)). In some cases, the ability of a composition described herein to bind minerals to teeth or tooth components can be observed, for example, using scanning electron microscopy or prophylometry.

A composition provided herein can have a low risk of toxicity to the person using the composition, can contain one or more polypeptides of human origin, can contain one or more polypeptides naturally present in food or drink products, and/or can lack potentially toxic dyes.

The invention will be further described in the following examples, which do not limit the scope of the invention described in the claims.

### EXAMPLES

### Example 1

### DOPA Content of Mussel Adhesive Polypeptides

A UV difference assay was used to determine the DOPA content of recombinant mussel adhesive polypeptides (MAP 22.6 kDa, catalog #260012; MAP 37 kDa, catalog #260022, purchased from Amsbio) as well as commercially extracted, native mussel adhesive polypeptides (purchased from Cell-Tak and ACRO Biosystems). The recombinant polypeptides are single chain polypeptides that contain sequences from both MFP-1 and MFP-5.

In the UV difference assay, the difference in absorption is measured in two different solutions; 0.2 M borate buffer, pH 8.0, and 0.2 M HCl. L-DOPA forms complexes with borate that causes a shift in the absorption at pH 8.0. The difference in absorption between 0.2 M HCl and 0.2 M borate buffer is greatest at 292 nm, the wavelength at which the samples are measured. A sample containing an unknown amount of DOPA is diluted into 0.2 M borate buffer or 0.2 M HCl using the same dilution factor. The absorption is measured and the absorption difference is calculated. The DOPA content is calculated using Lambert-Beer' Law with an extinction coefficient of 1885 M⁻¹ cm⁻¹. The assay is measured at room temperature. It was found that a good range to measure the DOPA content was between 25 to 200 μM L-DOPA.

The recombinant 22.6 kDa mussel adhesive polypeptide had a DOPA content of 10.8%. The recombinant 37 kDa mussel adhesive polypeptide had a DOPA content of 3.9%. The mussel adhesive polypeptides from ACRO and Cell-Tak precipitated in the assay so their DOPA content was not measured by this method.

### Example 2

### Oxidative Stability of DOPA Residues

Experiments were performed to test the susceptibility of DOPA residues to oxidation at different pH values to ensure that the subsequent conjugation reactions did not result in the oxidation of the DOPA residues. Both a colorimetric assay and a UV assay (as described in Example 1) were used to monitor the stability of the DOPA residues. In the colorimetric assay, a stock solution of 10 mM DOPA was prepared in 1% acetic acid and used to prepare a standard curve from 500 μM to 0 μM DOPA. The samples were diluted 1:10 into a variety of different buffers and 1 M NaOH was used to insure that the pH of the buffer solution remained at the desired value. The samples were incubated at room temperature and samples were taken every couple of hours and diluted into 1% acetic acid to determine the extend of DOPA oxidation.

When L-DOPA is oxidized in the presence of tetrazolium blue (TZB), the TZB is reduced to its pink-purple colored di-formazan form in a non-aqueous medium. The maximum absorption is found at 525 nm. A master mix solution containing TZB is added to the standard curve samples and the unknown samples. The master mix has a final TZB concentration of 0.25 mg/ml in ethanol containing 0.01% w/v KOH. In some cases small amounts of NaOH were added to achieve a pH of 12 or higher. The assay is incubated at 37 C, for 30 min., the samples are cooled back to room temperature, and their absorption at 525 nm is measured.

Samples were also inspected by eye and in some cases pictures of the oxidized samples were taken. Oxidation of DOPA was indicated by a brown to dark brown coloration of DOPA.

It was determined that DOPA was stable to oxidation for at least 24 hours in pH 5 acetate, pH 7 HEPES, and pH 8 borate buffer. In pH 8.5 and pH 9.0 bicarbonate buffers, most of the DOPA oxidized in 24 hours. Borate buffer at pH 8 and 8.5 prevented oxidation of DOPA residues. DOPA residues in this case are assumed to form a complex with the borate ion that is stable to oxidation.

### Example 3

### BFP—Stability at Acid pH Values

The stability of BFP in acid was assessed by incubating the protein (66 μg/ml) in phosphate buffered saline (PBS), 1% acetic acid (pH ˜2.7), 5% acetic acid (pH ˜2.4), acetate, pH 4, or acetate pH 5 for up to 72 hours, and then measuring the fluorescence. The BFP stock solution was at a concentration 4 mg/mL in PBS and was diluted in respective pH solution at ratio 1:60 v/v.

BFP was stable for several days at pH values 4. In contrast, fluorescence was lost in 1% acetic acid (pH ˜2.7) and 5% acetic acid (pH ˜2.4). When the same experiments were performed in the presence of 0.05% Tween 20, BFP was unstable at pH 4 but remained stable at pH values pH 5.

In BFP samples incubated with 1% acetic acid, over 50% of the fluorescence was restored by increasing the pH to 8. In contrast, in BFP samples incubated for 3 hours in 5% acetic acid, no activity was restored by increasing the pH.

### Example 4

### Binding of Different MAP Proteins to Teeth

In order to compare the binding of different mussel adhesive polypeptides to bovine teeth, the amount of protein bound per a fixed tooth surface area was determined. Because each tooth is unique in its size and shape, the surface area from tooth to tooth is different and it is difficult to calculate the surface area. A method was developed to standardize the surface area for each tooth by adhering a small polypropylene cup onto the tooth using silicone as a sealant. In this way the amount of protein bound to a fixed tooth surface area can be measured and is independent of tooth variations.

The binding of the mussel adhesive polypeptides described in Example 1 to bovine teeth was assessed. Bovine teeth were incubated with different MAPs for 2 hours at 37° C. using a range of different amounts of MAP from 15 μg to 0 μg (negative control). Different buffers were used to investigate binding at different pH values (carbonate buffer pH 9; 0.2 M MOPS pH 7; 0.2 M acetate buffer pH 5; 1% acetic acid pH 2.75). After binding, unbound protein was washed away with water, and the amount of protein bound was measured via a colorimetric assay (Bradford). Binding was observed with the recombinant 22.6 kDa polypeptide, the recombinant 37 kDa polypeptide, and the Cell-Tek sample, with the recombinant 22.6 kDa polypeptide having the best binding. Significant binding was not observed with the ACRO sample.

To establish how different tooth pre-treatments affected the binding of the mussel adhesive polypeptides, the bovine teeth were cleaned and pumiced. Two sets of teeth were further treated with either 5% EDTA, pH 7 or 5% citric acid, and the binding of the recombinant 22.6 kDa and 37 kDa polypeptides, and the Cell-Tak product were tested. The best binding was observed when the bovine teeth were pretreated with citric acid. The recombinant 22.6 kDa and 37 kDa polypeptides had similar binding after citric acid pretreatment. However, this is a harsh treatment and may not be useful for treatment of humans. For teeth pretreated with either EDTA or water, the recombinant 22.6 kDa polypeptide had the highest amount of binding. In another experiment, a small volume of mussel adhesive polypeptide was applied to bovine teeth using the silicone ring method, dried at 37° C. for about 90 minutes, and then extensively washed with water. A film could be seen as a circle in the middle of the teeth. The teeth were then brushed using a toothbrush and water. After brushing, the mussel adhesive polypeptide film could not be easily seen. To detect binding, teeth were stained with Coomassie blue (Coomassie Brilliant Blue R-250 staining solution from BioRad, Cat. No. 161-0436) for 1 min, washed in water, inspected for Coomassie stain, brushed with toothpaste and inspected again for Coomassie stain. The brushing step removed non-specifically bound Coomassie blue.

Using the Coomassie staining procedure, it was clear that the polypeptide remained on the teeth after brushing. The stained mussel adhesive polypeptide could not be removed from the teeth by brushing with toothpaste.

Other experiments were performed by adding small amounts of mussel adhesive polypeptides (MAP 22 kDa), MAP precursor (a recombinant MAP in which tyrosine residues are not converted to DOPA residues) or bovine serum albumin (BSA) in 1% acetic acid to bovine teeth and allowing the teeth to dry at 37° C. The samples were washed, stained with Coomassie Blue, washed again, photographed, and then brushed with toothpaste. MAP, MAP precursor, and BSA all bound to teeth after drying in 1% acetic acid, and remained on the teeth after brushing. In contrast, casein and BFP do not significantly bind to teeth under these conditions. Incubation for 1, 2, or 4 hours did not change the amount of binding. Similar results were observed with human teeth.

In another experiment, MAP and MAP Precursor in 1% acetic acid were incubated on teeth without drying for 3.5 hours. Only the MAP bound under these conditions, as determined by Coomassie staining, and remained after brushing. Qualitatively, it appears that MAP dried onto teeth is more resistant to brushing than MAP bound to teeth under wet conditions.

To determine the affect of pH, MAP was incubated on teeth without drying at pH 2.7 (1% acetic acid), pH 5.3 acetate buffer), and pH 7.3 HEPES buffer for 3.5 hours. After washing with water and drying, no film was visible on any tooth. Treatment with Coomassie resulted in strong staining for the 1% acetic acid treated teeth, weak staining for the pH 5.3 treated teeth, and no apparent staining for the pH 7 treated teeth.

### Example 5

### Preparation of Mussel Adhesive Polypeptide-BFP Conjugates

The 22.6 kDa mussel adhesive polypeptide (MAP) was activated for conjugation by thiolating the lysine residues using Traut's reagent (2-iminothiolane (IT)). The polypeptide (0.4 mg/ml) was incubated with 1.8 mM or 5 mM IT at pH 8 for 40 minutes at room temperature in the presence of sodium borate to protect the DOPA residues of the mussel adhesive polypeptide from oxidation. Two batches of MAP were produced with different numbers of thiols attached, MAP-Hi-SH (5 mM IT) and MAP-Lo-SH (1.8 mM IT).

BFP (1 mg/mL) was activated with two concentrations of maleimide (MAL-dPEG₄-NHS ester; Quanta BioDesign, catalog #10214; 0.3 mg/ml, 0.6 mg/ml). This resulted in two batches of BFP with different numbers of maleimides attached, BFP-Hi-MAL (0.6 mg/ml MAL-dPEG₄-NHS ester) and BFP-low-MAL (0.3 mg/ml MAL-dPEG₄-NHS ester).

A series of small scale (analytical amounts of protein) conjugation reactions were performed that contained the following:
Three reactions containing MAP-Lo-SH+BFP-Lo-MAL at MAP/BFP ratios of 3:1, 1:1, and 1:3
Three reactions containing MAP-Lo-SH+BFP-Hi-MAL at MAP/BFP ratios of 3:1, 1:1, and 1:3
Three reactions containing MAP-Hi-SH+BFP-Lo-MAL at MAP/BFP ratios of 3:1, 1:1, and 1:3
Three reactions containing MAP-Hi-SH+BFP-Hi-MAL at MAP/BFP ratios of 3:1, 1:1, and 1:3.

The reaction products were analyzed by SDS-PAGE. All of the MAP was converted to conjugate when using a MAP:BFP ratio of 1:3 and MAP-hi-SH+BFP-hi-MAL. When MAP: BFP ratios of 3:1 and 1:1 were used, lower amounts of conjugates were produced, with a slightly different molecular weight pattern observed on the gel.

In a medium scale conjugation ((1.4 mg-5.6 mg total protein), two MAP-BFP conjugates were prepared with a 1:1 MAP to BFP ratio and a 1:3 MAP:BFP ratio. A precipitate formed during the conjugation reactions and the yields of soluble conjugate were low. Precipitation also may have occurred in the small scale reaction described above, but the small volumes made any precipitate difficult to see. The soluble reaction products were analyzed by SDS-PAGE. The precipitate also was analyzed by SDS Page electrophoresis and appeared to contain a large polymer that did not enter the SDS gel.

### Example 6

### Binding of MAP Conjugates to Teeth

Bovine teeth were prepared for binding experiments by first cleaning them with a toothbrush and toothpaste and then pumicing them using a hand held dental polisher with a rubber cup. In certain experiments, teeth were incubated for 2 hours at room temperature with either 5% Disodium EDTA or 7.5% Citric Acid.

The precipitate from the 1:1 MAP/BFP medium scale reaction (Example 5) was converted to a cloudy suspension by pipetting the precipitate in pH 5 acetate buffer up and down multiple times. This suspension was dried onto the citric acid treated bovine teeth and formed a fairly homogeneous coating that was visible to the naked eye. This coating was not removed by extensive washing and showed strong fluorescence when illuminated with the 360 nm lamp.

The coating had a slight yellow tinge. It was determined that when the recombinant 22.6 kDa polypeptide was dissolved in acetic acid, the solution was yellow. In contrast, when the recombinant 37 kDa polypeptide was dissolved in acetic acid, the solution was clear. Since both of these products contain the same components (mfp1+mfp5), it is likely that there was a difference in their preparation. As such, the yellow tinge can be avoided by preparing the adhesive polypeptides under conditions in which the oxidation of the DOPA residues is minimized.

### Example 7

### Analysis of BFP-MAP Conjugate Precipitates Dried on Human Teeth

The re-suspended precipitates formed in the medium scale reactions between activated MAP and activated BFP (Example 5) were applied to human teeth, washed extensively with water, and then analyzed for whitening by photography in the presence of lighting that mimicked outdoor conditions. Two application methods were used. In the “silicone ring” method, the conjugate was added to a tooth area bounded by a silicone ring and dried. In the “submerged” method, the tooth was incubated in a conjugate suspension overnight.

Using the silicone ring method, the MAP conjugate formed a coating on the teeth that had a visible yellow tint. Although a significant amount of BFP:MAP conjugate bound to teeth, the yellow tint hid any whitening that may have occurred due to the immobilization of BFP on the tooth (in most cases, the D shades decreased rather than increased indicating that the surface was less white than before treatment, see Table 3). No coating was visible using the Submerged Method (see Table 2).

TABLE 2

| Tooth | BFP:MAP | Application | |
|---|---|---|---|
| id | Ratio | Method | D shades |
| 131 | 1:1 | Silicon ring | −2 |
| 132 | 1:1 | Silicon ring | −3 |
| 133 | 1:3 | Silicon ring | −1 |
| 134 | 1:3 | Silicon ring | 2 |
| 135 | Control | Silicon ring | 1 |
| 136 | 1:1 | Submerged | 0 |
| 137 | 1:3 | Submerged | 0 |
| 138 | Control | Submerged | 0 |

In order to obtain a semi-quantitative measure of BFP immobilized on the teeth, the values of blue light that were obtained after eliminating the red and green channels were calculated. This measurement is affected less by the yellow tint in the coating. The results are shown below in Table 3.

TABLE 3
BFP-MAP Conjugate and BFP-P-MAP Conjugate
Application: Measurement of Enhancement in
Blue channel Before and After Treatment

| Tooth | BFP:MAP | Application | Before | After | |
|---|---|---|---|---|---|
| id | Ratio | method | Blue | Blue | D blue |
| 131 | 1:1 | Silicon ring | 188.21 | 201.02 | 12.80 |
| 132 | 1:1 | Silicon ring | 212.14 | 219.42 | 7.27 |
| 133 | 1:3 | Silicon ring | 201.74 | 203.98 | 2.24 |
| 134 | 1:3 | Silicon ring | 227.92 | 230.67 | 2.75 |
| 135 | Control | Silicon ring | 207.53 | 206.15 | −1.38 |

The blue channel had significantly higher values for the 1:1 conjugate that was dried onto the teeth. The blue channel value is also higher than previously found for binding of the casein-BFP conjugate to pumiced teeth.

### Example 8

### Preparation of BFP Polymer-MAP Conjugate

BFP polymer was prepared and activated as follows. BFP was split into two batches; one batch was activated with Trauts reagents to add thiol groups to BFP and the second batch was activated with MAL-dPEG₄-NHS ester to add maleimide groups to BFP. The activation of BFP with Trauts reagent was done in 50 mM borate buffer pH 8.0 containing 2 mM EDTA and 0.05% Tween-20. The final concentration of iminothiolane was 45 mM. BFP was kept at 1 mg/ml. The reaction was incubated at RT for 40 min. Unreacted IT was removed from the reaction by gel filtration in 50 mM phosphate buffer, 2 mM EDTA, 0.05% Tween-20 pH 7.0. In the maleimide reaction, BFP was at a concentration of 1 mg/ml and MAL-dPEG₄-NHS ester was added to yield a final concentration of 3 mg/ml in a buffer of 50 mM phosphate, 2 mM EDTA, 0.05% Tween-20. The reaction was incubated for 40 min at room temperature. Unreacted MAL-dPEG₄-NHS ester was separated from BFP-M by gel filtration chromatography on a Sephadex G25 column (GE Healthcare) in 5 mM MES, 2 mM EDTA, 0.05% Tween-20, pH 6.

The two activated BFP species were then reacted together to form poly-BFP. Activated BFP species were combined at a 1:3 ratio of BFP-maleimide and BFP-SH and incubated for 1 hour at room temperature in 50 mM phosphate buffer, pH 7.0, 2 mM EDTA, 0.05% Tween-20. The reaction was quenched with NMM. The sample was concentrated and applied to a size exclusion column (Superdex 200, GE Healthcare) to separate poly-BFP of different sizes and to remove unreacted BFP.

In order to react poly-BFP with MAP-SH, a BFP-poly fraction with molecular weight of more than 220 kDa was used. Polymeric BFP was activated by reaction with 0.33 mg/mL MAL-dPEG₄-NHS ester in a 50 mM phosphate buffer containing 2 mM EDTA and 0.05% Tween 20 at pH 7 and, after unreacted reagent was removed by gel filtration, reacted with MAP-SH (activated with 5 mM iminothiolane) at different MAP/BFP ratios (MAP: BFP; 4:1; 2:1; 1:1; 1:2; 1:4). It was found that ˜75% of the BFP precipitated at high MAP/BFP ratios of 1:2 or 1:4. Under these conditions, there was sufficient MAP to coat the polymeric BFP and further polymerization or aggregation followed by precipitation did not occur. Increasing the relative amount of BFP in the conjugation decreased the amount of BFP in the precipitate.

### Example 9

### Successive Binding of MAP and BFP to Teeth (Double Sided Tape)

Binding of the native MAP (ACRO Biosystems; referred to as MAP1) or recombinant MAP (Amsbio; 22.6 kDa referred to as MAP2; 37 kDa referred to as MAP3), and BFP to bovine teeth was assessed by applying the MAP followed by the BFP to teeth. The MAP were incubated in sodium borate (SB) buffer, pH 8.0 for 4 hours at 37° C. followed by incubation with BFP (1 mg/ml) overnight at 37° C. For MAP 1, the concentration was 20 μg in 1 mL; for MAP 2, the concentration was 80 μg in 1 mL; for MAP 3, the concentration was 80 μg in 1 mL. The teeth had no pretreatment and one set of teeth were pumiced to assess if there is any effect of pumicing the teeth. Binding was assessed after overnight incubation, washing and pictures of the teeth were taken for analysis (pictures of the same teeth were recorded before the experiment for data comparison). The results are shown in Table 4. Only minor whitening was observed.

TABLE 4

| Pretreatment | Composition | Before | After | d RGB | d Shades |
|---|---|---|---|---|---|
| Pumice | MAP1 + BFP | 205.962 | 222.34 | 16.378 | 5 |
| | MAP2 + BFP | 199.762 | 205.963 | 6.201 | 2 |
| | MAP3 + BFP | 194.64 | 202.938 | 8.298 | 3 |
| | MAP1 Control | 196.91 | 206.687 | 9.777 | 3 |
| | BFP Control | 185.361 | ** | | |
| No Pumice | MAP1 + BFP | 183.667 | 200.383 | 16.716 | 6 |
| | MAP2 + BFP | 190.141 | 201.548 | 11.407 | 4 |
| | MAP3 + BFP | 182.027 | ** | | |
| | MAP3 Control | 195.354 | 200.168 | 4.814 | 2 |
| | BFP Control | 200.231 | 206.186 | 5.955 | 2 |

** data cannot be calculated.

### Example 10

### Titanium Dioxide Particles

Titanium dioxide particles (20 nm) and MAP 37 were mixed at a 10:1 nanoparticle:MAP37 ratio and incubated in acetic acid for 2 hours at 37° C. Different amounts of the suspension were pipetted onto the dentin part of bovine teeth (tooth was silver stained), and incubated at 37° C. for about 30 min to dry the mussel adhesive polypeptide and nanoparticles on the teeth. The teeth were then washed with water and incubated in water on a gel shaker for 44 hours. The particles remained on the teeth after the water wash but were removed by brushing with a toothbrush in water.

### Example 11

### Preparation of a Peptide Containing 30% DOPA Residues

The following peptide was made by solid phase amino acid synthesis (Ohio Peptide): KYKGKGYKGG YKGKYYGKGK KYYYKYKGKG KYKYGKKKGK YKGKGYKYYG, where Y refers to L-DOPA (L-dihydroxyphenylalanine) (SEQ ID NO:21). This peptide contains 30% DOPA, 42% Lysine, and 28% Glycine, and is referred to as MAPtide herein. The sequence was adapted from amino acid 46-95 of the Mfp-5 sequence of Mytilis edulis (SEQ ID NO:6) with residues that are not Y, K, or G substituted with K or G.

The MAPtide was dissolved in 1% acetic acid to a concentration of 5 mg/mL and applied to bovine teeth under wet and dry binding conditions. For wet binding, the teeth were incubated with 10 μl of the peptide solution for 30 min at 37° C. over a reservoir of 1% acetic acid. A series of concentrations were tested ranging from 1000 μg/mL to 1.6 μg/mL. After brushing with toothpaste and staining the teeth with Coomassie Blue Dye, a strong stain was visible for all concentrations down to 8 μg/ml. A weak stain was still visible at 1.6 μg/ml. Under dry binding conditions, two concentrations (1000 μg/mL and 200 μg/mL) were tested. Ten μL of the peptide solution was applied on the teeth, dried over night at 37° C., and brushed with toothpaste. Again, a strong Coomassie stain was visible.

### Example 12

### Synthesis of Organic Polymers Substituted with DOPA

Poly DOPA was synthesized using three different polymers: polymethacrylic acid (PMA), poly[ethylene-co-(maleic anhydride) (PEMA), and poly[butadiene-co-(maleic anhydride) (PBMA).

Poly-L-DOPA-PMA polymers were synthesized using different concentrations of DOPA. 1-Ethyl-3-(3-dimethylaminopropyl) carbodiimide (EDC; 0.5 g) and N-hydroxysuccinimide (NHS; 0.5 g) were dissolved in a PBS solution containing 2.5 g of PMA (MW 9.5 kDa, Sigma) solution. After the solution was mixed for 4 h at room temperature, L-DOPA (0 g for PMA 1, 0.25 g for PMA2 and 0.75 g for PMA3) was added to the respective polymer solutions (Table 5). The reaction volumes were adjusted to 10 ml with PBS and the reactions were stirred for approximately 2 h. The precipitated reaction products were then washed well with ethanol, the precipitated poly(L-DOPA) was dried, and the product stored in vacuum desiccator.

Poly-L-DOPA-PEMA and Poly-L-DOPA-PBMA polymers were synthesized as follows. In a round bottom flask attached with a cold water condenser, 0.5 g of each polymer poly[ethylene-co-(maleic anhydride)] 1:1 (PEMA, MW, 400 kDa, Polysciences) and poly[butadiene-co-(maleic anhydride)] 1:1 (PBMA, MW 10-15 kDa, Polysciences) was added to 0.65 g DOPA in 50 ml ethanol. The reactions were refluxed at 70-90° C. on hot plates in water or oil bath for 12 h with constant stirring. After 12 h, the reactions were cooled to room temperature and the precipitated polymers were dried and stored in a vacuum desiccator (Table 5).

TABLE 5
Synthesis of poly-L-DOPA with PMA, PEMA and PBMA.

| | Molecular | | | | |
|---|---|---|---|---|---|
| | wt | | | | % |
| | (average) | | | Yield | Dopa |
| Polymer | kDa | Color | Solubility | grams | by wt |
| PMA1 | 9.5 | White | Water | 2.11 | 0 |
| PMA2 | 11-12 | White | Water | 2.23 | 9.91% |
| PMA3 | 12-13 | White | Water | 2.56 | 22.24% |
| PBMA | 20-25 | Off white | Alkaline/0.5MHCl | 0.767 | 33.4% |
| PEMA | 600 | White | Alkaline/0.2MHCl | 0.964 | 39.9% |

### Example 13

### Initial Binding Studies of DOPA Substituted Organic Polymers

The binding of the polymers from Example 2 to bovine teeth was assessed as follows. Ten μl of PMA 1, 2, and 3 (5 mg/ml) in 1% acetic acid, or PEMA or PBMA in 0.2M HCl at 2 mg/ml were applied to each tooth under dry conditions and incubated overnight. The teeth were stained with Coomassie blue as described above, washed with water, and brushed extensively with toothpaste. Strong binding was observed of all polymers containing DOPA. Very low binding was observed in control reactions using PMA with no DOPA (PMA 1).

In a related experiment, the PMA polymers in 1% acetic acid were dried on teeth, washed with water, and then incubated in pH 8.5 bicarbonate buffer overnight. For PMA2 and PMA3, the bicarbonate solution turned brown because of dissolved DOPA but the teeth remained white. These teeth showed strong Coomassie staining after brushing with toothpaste. Again, PMA1 showed no significant staining.

### Example 14

### Preparation of MAPtide-BFP Conjugates

MAPtide (1 mg/mL) (see Example 11) was activated for conjugation by incubation with 1 mg/mL of Maleimide-dPEG®4-NHS ester (Quanta Biodesign) for 40 min at RT in 50 mM sodium phosphate, 2 mM EDTA, 0.05% TWEEN-20, pH 7.0. The reaction mixture was concentrated using a 3 kDa spin filter (Millipore) and purified in 5 mM MES (2-(N-morpholino)ethanesulfonic acid), 2 mM EDTA, and 0.05% TWEEN-20, pH 6 with a gel filtration column (PD-10; GE Healthcare) to remove the activated protein from un-reacted reaction components.

BFP (1 mg/mL) was activated for conjugation by incubation with 3.3 mg/mL of SPDP-dPEG®12-NHS ester (Quanta Biodesign) for 40 min at room temperature in 50 mM sodium phosphate, 2 mM EDTA, 0.05% TWEEN-20, pH 7.0. The thiol group was activated with 10 mM DTT (dithiothreitol). The reaction mixture was concentrated and then purified on a PD-10 column in 5 mM MES, 2 mM EDTA, and 0.05% TWEEN-20, pH 6.

For the conjugation reaction, activated MAPtide and activated BFP were combined at molar ratios of between 3:1 and 5:1 in 50 mM sodium phosphate, 2 mM EDTA, and 0.05% TWEEN-20, pH 7.0 and incubated for 60 min at room temperature. The reaction products were analyzed by SDS PAGE electrophoresis. The conjugates were dialyzed into 5 mM acetate buffer, pH 5 using a 10 kDa dialysis membrane (Spectrum) to remove un-reacted MAPtide and to stabilize DOPA residues.

### Example 15

### Preparation of Poly(MAPtide-BFP)

The BFP-MAPtide conjugate, prepared as described in Example 14, was separated into two fractions. One fraction of BFP-MAPtide (0.6 mg/mL MAPtide) was activated with 3.3 mg/ml Maleimide-dPEG®4-NHS ester as described in Example 14 for the activation of MAPtide. The other fraction of MAPtide-BFP (0.6 mg/mL MAPtide) was activated with 3.3 mg/ml SPDP-dPEG®12-NHS ester as described in Example 14 for the activation of BFP.

The polymerization reaction was started by combining the Maleimide activated BFP-MAPtide conjugate with the SPDP activated MAPtide-BFP conjugate at a ratio of 3:1. The reaction was incubated for 60 min at room temperature in 50 mM sodium phosphate, 2 mM EDTA, 0.05% TWEEN-20, pH 7.0. The reaction mixture was analyzed by SDS PAGE electrophoresis. The polymer was concentrated and applied to a Superdex 200 16/60 gel filtration column (GE Healthcare) that was equilibrated in 5 mM acetate buffer at pH 5. The column fractions were separated into large, medium, and small molecular weigh polymers as determined by the SDS-PAGE analysis of the fractions.

### Example 16

### Preparation of MAPtide-PolyBFP

To prepare a MAPtide-PolyBFP conjugate, a BFP polymer was prepared, and then the MAPtide and the BFP-polymer were activated and conjugated to each other.

Preparation of PolyBFP

A solution of BFP (1 mg/ml) was activated for conjugation with 3.3 mg/mL of Maleimide-dPEG®4-NHS ester as described in Example 14 for the activation of MAPtide. A second solution of BFP (1 mg/ml) was activated with 3.3 mg/mL of SPDP-dPEG®12-NHS ester as described in Example 14 for the activation of BFP.

BFP was polymerized by combining the maleimide and SPDP activated solutions of BFP in equal amounts at room temperature for 60 min in 50 mM sodium phosphate, 2 mM EDTA, 0.05% TWEEN-20, pH 7.0. The polymerization was analyzed by SDS PAGE electrophoresis. To purify the polymerized BFP, the reaction was concentrated and applied to a Superdex 200 16/60 gel filtration column equilibrated in 5 mM sodium acetate buffer pH 5.

Preparation of MAPtide-PolyBFP Conjugate

PolyBFP (1 mg/mL) was activated with 3.3 mg/mL SPDP-dPEG®12-NHS ester (Quanta Biodesign) as described in Example 14 for the activation of BFP. MAPtide (1 mg/mL) was activated for conjugation with 1.1 mg/mL of maleimide-dPEG®4-NHS ester as described in Example 14 for the activation of MAPtide.

The conjugation of activated PolyBFP with activated MAPtide was performed in a reaction that contained a 6 fold molar excess of MAPtide. The conjugation was performed at RT for 60 min in 50 mM sodium phosphate, 2 mM EDTA, 0.05% TWEEN-20, pH 7.0. The conjugation reaction was analyzed on SDS PAGE electrophoresis. The polymer-conjugate was dialyzed into 5 mM sodium acetate buffer at pH 5 to stabilize the polymer conjugate and remove un-reacted MAPtide.

### Example 17

### Preparation of Hydroxyapatite (HA) Complexes with MAPt Poly(MAPtide-BFP) and MAPtide-PolyBFP

Each of the different conjugates of Examples 14-16 (MAPtide-BFP, Poly(MAPtide-BF), and MAPtide-PolyBFP) was incubated at concentration of 0.2 mg/mL (MAPtide content) with 1.25 mgiinL HA in 0.1 M MES, pH 6, for 1 hour at room temperature on a platform shaker. After incubation, the reactions were centrifuged at 750×g at 4° C. for 2 min. Excess conjugate was removed from the precipitate and the HA complex was resuspended in 1 ml 0.1 M acetate buffer, pH 5. The solution was centrifuged again at 750×g at 4° C. for 1 min and the supernatant solution was discarded. The washing step was repeated two times. Finally, the HA complex was resuspended in 0.1 M acetate buffer, pH 5 at final concentration of 50 mg/ml of HA and used for treating teeth.

### Example 18

### Applying MAPtide-BFP/HA Complexes to Human Teeth

The complexes of Example 17 were used to assess whitening of human teeth. Extracted human teeth were sterilized by autoclaving and prepared for testing by a cleaning cycle of brushing, pumicing and brushing again in the laboratory. Teeth were brushed using toothpaste and a manual tooth brush and pumicing was done using professional polishing device and dental pumice.

In the experiments where saliva treated teeth were used, teeth were incubated in fresh human saliva for at least 2 hours to reform the pellicle, The teeth then were air dried and assigned a shade using a shade guide (see below). The teeth were placed facing upwards on playdough on a horizontal surface for application of the whitening reagent.

In some experiments, bleached human teeth were stained with tea or coffee before the complex was applied. The teeth were cleaned by brushing before the staining procedure. One bag of tea (Lipton®) and 2 g of instant coffee (Nescafe®) were dissolved together in 20 mL of hot DI water. The solution was allowed to cool at room temperature and made 0.02% sodium azide to prevent bacterial growth. Finally about 20 teeth were incubated in the solution for 30 days at 37° C. After 30 day of incubation the teeth appeared stained. The teeth then were removed from the staining solution and rinsed thoroughly with water. These teeth were brushed, pumiced and brushed before using them in a whitening experiment.

Between 5 and 10 teeth were used for each experimental condition. The MAPtide-BFP/HA slurry (or Poiy(MAPtide-BFP)/HA and MAPtide-PolyBFP/HA slurry) was mixed well before each application to prevent the HA particles from settling to the bottom of the tube. An aliquot of 10 μL was gently applied to the surface of each tooth and the reagent was spread over the entire surface of the tooth using a pipette tip. In some cases, reagent was spread on the surface of the tooth using a flow of air. The reagent was kept in contact with the tooth for 5-15 min at room temperature and, in some cases, it was dried on the surface of the tooth using a slow air flow. Each tooth then was washed with water and brushed to remove excess Maptide-BFP/HA Reagent from the teeth. Teeth were air dried and analyzed for enhanced whitening using a shade guide (see below).

Analysis of the shade improvement was done using two methods, in the first method, teeth were place on a holder in a light box that mimics natural daylight. Before and after treatment pictures were recorded for each tooth in the light box using a camera and manual exposure settings. The pictures were analyzed for shade improvement using Java image processing program ImageJ (see the world wide web at rsbweb.nih.gov/ij/). In the second method, teeth were analyzed for enhanced shade whitening using the VITA 3D-Master® shade system (see world wide web at vident.com/products/shade-management/vita-3d-master-shade-guide). The shade guide was used according to the manufactures instructions. Both methods compare difference in lightness (value), color saturation (chroma) and hue before and after treatment. The average shade change and the standard deviation of the shade change was calculated for each experiment.

### Example 19

### Tooth Whitening of Naturally Stained Human Teeth Using MAPtide-BFP/HA

Naturally stained human teeth were brushed, pumiced and brushed as described in Example 18. The shade of each tooth was determined and the teeth were incubated with the MAPtide-BFP/HA reagent as described in Example 18. The teeth then were rinsed, brushed with toothpaste, rinsed again, and dried before the final shade was determined

Four separate whitening experiments were performed using 20 total test teeth and 3 untreated control teeth. The test teeth showed an increase in whiteness of 6.5±3.5 shades while the control teeth showed no whitening. There was tooth to tooth variation, as indicated by the standard deviation of the results. However, 17 of the 20 treated teeth showed an increase in whiteness of ≧2 shades.

### Example 20

### Comparison of HA, MAPtide/HA, and MAPtide-BFP/HA as Tooth Whitening Agents

Sets of extracted human teeth were prepared and treated with either HA, MAPtide/HA, or MAPtide-BFP/HA as described in Example 18. The results are shown in Table 6. In each case, eight teeth were treated with the reagent and one or two teeth were untreated controls.

TABLE 6
Whitening Results with Different Tooth Whitening Agents

| | | | |
|---|---|---|---|
| | Shade before | Shade after | Shade number |
| Tooth number | whitening | whitening | improvement |

A. Shade Guide Analysis of Teeth Treated with HA

| | | | |
|---|---|---|---|
| 472 | 5M2 | 4M3 | 4 |
| 473 | 5M3 | 5M3 | 0 |
| 474 | 5M2 | 4M3 | 4 |
| 475 | 5M2 | 5M2 | 0 |
| 476 | 5M3 | 5M2 | 1 |
| 477 | 4M2 | 4M2 | 0 |
| 479 | 4M1 | 4M1 | 0 |
| 480 | 4M1 | 4M1 | 0 |
| | | Average (8 teeth) | 1 ± 2 |
| 471 (Control) | 5M1 | 5M1 | 0 |
| 478 (Control) | 5M1 | 5M1 | 0 |

B. Shade Guide Analysis of Teeth Treated with MAPtide/HA

| | | | |
|---|---|---|---|
| 481 | 4M1 | 4M1 | 0 |
| 482 | 4M1 | 4M2 | −1 |
| 483 | 5M1 | 3M3 | 10 |
| 484 | 5M3 | 5M1 | 2 |
| 485 | 5M1 | 4M3 | 3 |
| 488 | 4M1 | 4M1 | 0 |
| 489 | 5M3 | 4M1 | 7 |
| 490 | 3M2 | 3M1 | 1 |
| | | Average (8 teeth) | 3 ± 4 |
| 486 (Control) | 1M2 | 1M2 | 0 |
| 487(Control) | 2M1 | 2M1 | 0 |

C. Shade Guide Analysis of Teeth Treated with MAPtide-BFP/HA

| | | | |
|---|---|---|---|
| 491 | 4M2 | 3M2 | 5 |
| 492 | 4M1 | 3M1 | 7 |
| 494 | 5M3 | 4M2 | 6 |
| 495 | 4M1 | 3M1 | 7 |
| 496 | 5M1 | 4M2 | 4 |
| 497 | 5M1 | 3M3 | 10 |
| 498 | 5M2 | 4M3 | 4 |
| 499 | 5M1 | 4M3 | 3 |
| | | Average (8 teeth) | 6 ± 2 |
| 493(control) | 3M1 | 3M1 | 0 |

As shown in Table 6, the average shade improvement was 1±2 for HA; 3±4 for MAPtide/HA; and 6±2 for MAPtide-BFP/HA. The data indicate that HA alone has no significant effect. The whitening shade change obtained with MAPtide-BFP/HA is twice that obtained with MAPtide/HA. The whitening obtained using MAPtide-BFP/HA was, by visual observation, significantly greater than the whitening obtained by MAPtide-BFP without HA.

### Example 21

### Whitening Obtained with Saliva Coated Teeth

Naturally stained human teeth were brushed, pumiced and brushed as described in Example 18. The teeth then were incubated in fresh human saliva for 2 hours at room temperature and then rinsed before treatment with MAPtide-BFP/HA. The shade of each tooth was determined as previously described. Of the seven saliva coated teeth tested, five increased by ≧2 shades of whitening. The average increase in whitening was 3±2 shades. There was no change in the shades of the two untreated control teeth.

### Example 22

### Whitening Obtained with Teeth Stained with Tea+Coffee

Bleached teeth were incubated at 37° C. in a concentrated solution of tea and coffee as described in Example 18. The teeth were brushed, pumiced, and brushed again before treatment with MAPtide-BFP/HA. Seven of the nine teeth in this experiment showed an increase of whitening of two shades or more. The average increase in whiteness was 4±3 shades. The two untreated control teeth showed no increase in whitening.

### Example 23

### Tooth Whitening Obtained with Poly(MAPtide-BFP)/HA

Naturally stained teeth were brushed, pumiced, and brushed and treated with Poly(MAPtide-BFP)/HA as described in Example 18. Five of the five teeth tested showed an increase in whitening of ≧2 shades. The average increase in whitening was 7±4 shades. One control tooth had no increase in whitening and a second control tooth had 1 shade of whitening.

### Example 24

### Tooth Whitening Obtained with MAPtide-PolyBFP/HA

Naturally stained teeth were brushed, pumiced, and brushed and treated with MAPtide-PolyBFP/HA as described in Example 18. Five of the eight teeth tested showed an increase in whitening of 2 shades. The average increase in whitening was 4±4 shades. One control tooth had no increase in whitening and a second control tooth had 1 shade of whitening.

### Example 25

### Preparation of a 10-25 Amino Acid Peptide Containing 30% DOPA Residues

A peptide of 25 amino acids (YKGKG KYKYGKKKGK YKGKGYKYYG where Y refers to L-DOPA) along with multiple shorter fragments from this sequence were made by sold phase peptide synthesis. The peptides were conjugated to BFP using the methods described in Example 14 and then complexed with HA and applied to teeth using the methods described in Examples 17 and 18. The whitening obtained using this material was significantly less than the whitening obtained using MAPtide-BFP/HA which contains a larger MAPtide.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising an adhesive molecule and a whitening particle or remineralization particle, said adhesive molecule comprising a plurality of 3,4-dihydroxyphenyl-L-alanine (DOPA) residues and having the ability to interact with or bind to a tooth, a tooth component, or inorganic dental material.

2-68. (canceled)


SEQUENCE LISTING (30 sequences)

SEQ ID NO: 1 - DNA, 743 bp, Artificial Sequence
  encodes soluble -modified blue fluorescent protein
ggatccaaggagatataacaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctctcatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagagctc

SEQ ID NO: 2 - protein, 238 aa, Artificial Sequence
  blue fluorescent protein
MSKGEELFTGVVPILVELDGDVNGHKFSVSGEGEGDATYGKLTLKFICTTGKLPVPWPTLVTTFSHGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 3 - protein, 234 aa, Artificial Sequence
  blue fluorescent protein
MSEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 4 - DNA, 231 bp, Mytilus galloprovincialis
agttctgaagaatacaaaggtggttattacccaggcaatacttaccactatcattcaggtggtagttatcacggatccggctatcatggaggatataagggaaagtattacggaaaggcaaagaaatactattataaatataaaaacagcggaaaatacaagtatctgaagaaagctagaaaataccatagaaagggttacaagaagtattatggaggtggtagcagttag

SEQ ID NO: 5 - protein, 76 aa, Mytilus galloprovincialis
SSEEYKGGYYPGNTYHYHSGGSYHGSGYHGGYKGKYYGKAKKYYYKYKNSGKYKYLKKARKYHRKGYKKYYGGGSS

SEQ ID NO: 6 - protein, 98 aa, Mytilus edulis
MKLSCIVLVLFLVTLAAYSDVGSSSSEEYKGGYYPGNAYHYHSGGSYHGSGYHGGYKGKYYGKAKKYYYKYKNSGKYKYLKKARKYHRKGYKYYGGSS

SEQ ID NO: 7 - protein, 96 aa, Mytilus californianus
MKLSCVVLVLFLVTLAACIDVGSGYDGYSDGYYPGSAYNYPSGSHGYHGHGYKGKYYGKGKKYYYKYKRTGKYKYLKKARKYHRKGYKKYYGGGSS

SEQ ID NO: 8 - protein, 72 aa, Artificial Sequence
  chimeric mussel adhesive polypeptide
MNNTSVSVLVALVLIGSFAVQSDAADYYGPNYGPPRRYGGGNYNRYNRYGRRYGGYKGWNNGWNRGRRGKYW

SEQ ID NO: 9 - protein, 78 aa, Mytilis californianus
MNKFSVTVLLALVLIGFFAVQSDAGYGYDLGYDYYKGYNNPWPYGGKYNGYKGYPRGYGWNKGWNKGWNKGRWGRKYY

SEQ ID NO: 10 - protein, 70 aa, Mytilus galloprovincialis
MNNISVAVLVALVLIGSFAVQSDAADYYGPKYGPPRRYGGGNYNRYGRRYGGYKGWNNGWKRGRWGRKYY

SEQ ID NO: 11 - protein, 565 aa, Mytilus edulis
MEGIKLNLCLLCIFTFDVLGFSNGNIYNAHVSSYAGASAGAYKKLPNAYPYGTKPEPVYKPVKTSYSAPYKPPTYQQLKKKVDYRPTKSYPPTYGSKTNYLPLAKKLSSYKPIKTTYNAKTNYPPVYKPKMTYPPTYKPKPSYPPTYKSKPTYKPKITCPPTYKAKPSYPPTYKPKKTYPPTYKPKVTYPPTYKPKPSYPPIYKSKPTYKPKITYPPTYKAKPSYPPTYKAKPSYPPTYKAKPTYKAKPTYPSTYKAKPTYPPTYKAKPSYPPTYKAKPSYPPTYKAKPTYIAKPSYPPTYKAKPSYPPTYKAKPSYPPTYKAKSSYPPTYKAKPTYKAKPTYPSTYKAKPSYPPTYKAKPTYKAKPTYPSTYKAKPTYPSTYKAKPSYPPTYKPKISYPPTYKAKPSYPSTYKAKSSYPPTYKAKPSYPPTYKAKPTYKAKPTYPSTYKAKPTYKAKPTYPPTYKAKPSYPPTYKPKPSYPPTYKSKSSYPSSYKPKKTYPPTYKPKLTYPPTYKPKPSYPPSYKPKITYPSTYKLKPSYPPTYKSKTSYPPTYNKKISYPSQY

SEQ ID NO: 12 - protein, 732 aa, Mytilus edulis
MEGIKLNLCLLCIFSCDVFALSNGFIHNAYGSAYAGASAGAYKPLPGSYGSKHVPVYKPMNKIPTSYISKKSYPAPYKPKGYHPTNSYQPTYGSKTNYPPIYKPVAKKLSSYKAIKTTYLVYKAKTSYPPVYKHKITNPPTYKPKITYPPTYKPKPSYPPTYKPKPSYPPTYKAKKTYPSTYKPKPSYPPTYKPKITYPPTYKPKPSYPPSYKAKKSYPSTYKPKPSYPPTYKAKKTYPSTYKPKPSYPPTYKPKITYPPTYKPKPSYPPSYKAKKSYPSTYKPKPSYPPTYKPKITYPPTYKPKPSYPPTYKAKKTYPPTYKPKITYPPTYKPKPSYPTSYKSKKTYPPTYKPKITYPPTYKPKPSYPPSYKPKITYPPTYKPKKSYPPAYKSKASYPPSYQPKKTYLPSYKPKKTYPPTYKRKISYPPTYKTKPSYPSSYKRKTSYPSTYKRKTSYPPTYKPKISYPSTYKTKPSYPPTYKAKKTYPPTYKPKITYPPTYKPKPSYPTSYKSKKTYPPTYKPKITYPPTYKPKPSYPPSYKPKITYPPTYKPKKSYPPAYKSKASYPPSYQPKKTYLPSYKPKKTYPPTYKRKISYPPTYKTKPSYPASYKRKTSYPSTYKRKTSYPPTYKPKISYPSTYKTKPSYPPTYKPKPSYASSYKPKIRYPPTYKPKPSYASSYKPKITYPPTYKPKISYPPTYKPKITYPPSYKPKISYLPAYKPKISYPSQY

SEQ ID NO: 13 - protein, 10 aa, Artificial Sequence
  motif of mussel adhesive polypeptide
AKPSYPPTYK

SEQ ID NO: 14 - protein, 10 aa, Artificial Sequence
  motif of mussel adhesive polypeptide
PKISYPPTYK

SEQ ID NO: 15 - protein, 42 aa, Artificial Sequence
  motif of mussel adhesive polypeptide
TDKAYKPNPCVVSKPCKNRGKCIWNGKAYRCKCAYGYGGRHC

SEQ ID NO: 16 - protein, 48 aa, Artificial Sequence
  motif of mussel adhesive polypeptide
ADYYGPNYGPPRRYGGGNYNRYNRYGRRYGGYKGWNNGWNRGRRGKYW

SEQ ID NO: 17 - protein, 10 aa, Artificial Sequence
  motif of mussel adhesive polypeptide
HVHTHRVLHK

SEQ ID NO: 18 - protein, 11 aa, Artificial Sequence
  motif of mussel adhesive polypeptide
DDHVNDIAQTA

SEQ ID NO: 19 - protein, 73 aa, Artificial Sequence
  motif of mussel adhesive polypeptide
SSEEYKGGYYPGNAYHYSGGSYHGSGYHGGYKGKYYGKAKKYYYKYKNSGKYKYLKKARKYHRKGYKYYGGSS

SEQ ID NO: 20 - protein, 99 aa, Artificial Sequence
  motif of mussel adhesive polypeptide
GGGNYRGYCSNKGCRSGYIFYDNRGFCKYGSSSYKYDCGNYACLPRNPYGRVKYYCTKKYSCPDDFYYYNNKGYYYYNDKDYGCFNCGSYNGCCLRSGY

SEQ ID NO: 21 - protein, 50 aa, Artificial Sequence
  polymer containing DOPA residues
KXKGKGXKGGXKGKXXGKGKKXXXKXKGKGKXKXGKKKGKXKGKGXKXXG

SEQ ID NO: 22 - protein, 50 aa, Artificial Sequence
  polymer containing DOPA residues
KXKKKKXKKKXKKKXXKKKKKXXXKXKKKKKXKXKKKKKKXKKKKXKXXK

SEQ ID NO: 23 - protein, 50 aa, Artificial Sequence
  polymer containing DOPA residues
SXHGSGXHGGXKGKXXGKAKKXXXKXKNSGKXKXLKKARKXHRKGXKXXG

SEQ ID NO: 24 - protein, 50 aa, Artificial Sequence
  polymer containing DOPA residues
SXSSSSXSSSXKSKXXSKSKKXXXKXKSSSKXKXSKKSSKXSSKSXKXXS

SEQ ID NO: 25 - protein, 50 aa, Artificial Sequence
  polymer containing DOPA residues
GXSGKNXHGSXKGKXXHKHKKXXXKXKLLHKXKXGKKGNKXGGKGXKXXH

SEQ ID NO: 26 - protein, 50 aa, Artificial Sequence
  polymer containing DOPA residues
KXKXKXXKXXXKXKXXXKXKKXXXKXKXKXKXKXXKKKXKXKXKXXKXXX

SEQ ID NO: 27 - protein, 50 aa, Artificial Sequence
  polymer containing DOPA residues
KXKGKGGKGGXKGKGXGKGKKGXGKXKGKGKGKXLKKKGKGKGKGXKGXG

SEQ ID NO: 28 - protein, 50 aa, Artificial Sequence; identical to SEQ ID NO: 22
  polymer containing DOPA residues

SEQ ID NO: 29 - protein, 50 aa, Artificial Sequence
  polymer containing DOPA residues
KKGGXXKGKXKXGKKKKGGXXGGXXXGKKKXGKXXXXXKGKXXGXKXGKK

SEQ ID NO: 30 - protein, 50 aa, Artificial Sequence
  polymer containing DOPA residues
XXXXXXXXXXXXXXXXXXXXXXXXXXXXXXXXXXXXXXXXXXXXXXXXXX